(19) United States
(12) Patent Application Publication
(10) Pub. No.: US 2023/0008847 A1
(43) Pub. Date: Jan. 12, 2023

(54) ODOR PRESENTATION MODULE, ODOR PRESENTATION DEVICE, ODOR PRESENTATION SYSTEM, AND ODOR PRESENTATION METHOD

(71) Applicant: SONY GROUP CORPORATION, TOKYO (JP)

(72) Inventors: MITSUHIRO KAWANISHI, TOKYO (JP); KAZUHIKO MIYAHARA, TOKYO (JP); YUSAKU KATO, TOKYO (JP); YOSHIO GOTO, TOKYO (JP); AKIHIRO NAKATA, TOKYO (JP); HISATAKA IZAWA, TOKYO (JP)

(21) Appl. No.: 17/783,538

(22) PCT Filed: Dec. 15, 2020
(86) PCT No.: PCT/JP2020/046634

§ 371 Date: Jun. 8, 2022

(30) Foreign Application Priority Data
Dec. 19, 2019 (JP) 2019-229238
Jun. 29, 2020 (JP) 2020-111221

(51) Int. Cl. A61L 9/12; A63F 13/28; A63J 25/00; F24F 1/008; G06F 3/01

(52) U.S. Cl. CPC A61L 9/122; A63F 13/28; A63J 25/00; F24F 1/008; G06F 3/01; A61L 9/125; A61L 2209/133

(57) ABSTRACT

An object of the present technology is to provide a technology that enables control of an odor. The present technology provides an odor presentation module including a first opening that first airflow containing an odor exits, and a second opening that second airflow exits, in which the first opening and the second opening are provided so that movement of the first airflow exiting the first opening is able to be controlled by the second airflow. The present technology also provides an odor presentation device and an odor presentation system including the odor presentation module. Further, the present technology provides an odor presentation method including a first airflow generation step of emitting first airflow containing an odor from a first opening, and a second airflow generation step of generating second airflow exiting a second opening or second airflow entering the second opening, in which movement of the first airflow exiting the first opening is controlled by the second airflow.

### TECHNICAL FIELD

The present technology relates to an odor presentation module, an odor presentation device, an odor presentation system, and an odor presentation method. More specifically, the present technology relates to an odor presentation module, an odor presentation device, an odor presentation system, and an odor presentation method capable of controlling an odor that is presented to a user.

### BACKGROUND ART

In recent years, presenting an odor according to a video has been performed. For example, an odor is presented to a person watching a movie at a predetermined timing. Several technologies for presenting such odors have been proposed so far.

For example, PTL 1 below discloses an olfactory display that presents a scent in a temporally and spatially limited range, and includes a housing having an injection port, a plurality of scent chambers formed by partitioning an internal space of the housing with a partition wall and having an intake port and a scent outlet, a scent passage that causes each of the scent outlets to communicate with the injection port, a solid scent source stored in the scent chamber, a plurality of wind sources that are provided in the respective scent chambers and send air from the intake port into the scent chamber using a diaphragm having a piezoelectric element, and an auxiliary wind source that is provided in the housing separately from the wind source and accelerates a scent sent from the scent chamber by the wind source in a scent injection direction using the diaphragm having the piezoelectric element.

### CITATION LIST

### Patent Literature

[PTL 1]

JP 2014-230632 A

### SUMMARY

### Technical Problem

As described above, an odor is presented according to a video. However, even when an odor associated with a video is presented to a viewer of the video, the viewer may perceive a sense of discomfort. This discomfort may be due to the way the odor is presented.

The impression that a human perceives about an odor varies greatly depending on the way the odor is presented. For example, a human to whom an odor is injected by an injection device such as a spray perceives wind pressure at the same time as perception of the odor, and in addition, a time in which the human perceives the odor is short. On the other hand, for example, for an odor from a sprayed scent, a human perceives only the odor without perceiving wind pressure, and in addition, a time in which the human perceives the odor is long. The impression that the human perceives greatly differs between a local odor like the former and a drifting odor such as the latter. Thus, the way an odor is presented is considered to be an important factor in expression of the odor.

Therefore, an object of the present technology is to provide a technology for controlling an odor that is presented to a human.

### Solution to Problem

The present technology provides an odor presentation module including:

a first opening that first airflow containing an odor exits; and

a second opening that second airflow exits,

wherein the first opening and the second opening are provided so that movement of the first airflow exiting the first opening is able to be controlled by the second airflow.

The odor presentation module may include a first airflow generation device, and an odor carrying portion that carries the odor, and

the odor presentation module may be configured such that airflow generated by the first airflow generation device and the odor included in the odor carrying portion form the first airflow.

The odor presentation module may further include a second airflow generation device, wherein the second airflow generation device may generate the second airflow.

The odor presentation module may be configured such that the first airflow is injected from the first opening.

The odor presentation module may be configured such that the first airflow and the second airflow come into contact with each other outside the odor presentation module.

The odor presentation module may be configured such that the first airflow and the second airflow come into contact with each other inside the odor presentation module.

The first opening may be in front of the second airflow generation device in a traveling direction of the second airflow.

The odor presentation module may be configured to be able to change the flow velocity of at least one of the first airflow and the second airflow.

The odor presentation module may be configured to be able to change a direction of the second airflow to a direction into the second opening.

Further, the present technology provides

an odor presentation module including:

an odor-containing airflow generation unit; and

a control airflow generation unit configured to control odor containing airflow generated by the odor-containing airflow generation unit,

wherein the odor-containing airflow generation unit includes a first airflow generation device, an odor carrying portion through which airflow generated by the first airflow generation device passes, and a first opening that first airflow containing an odor formed by passing through the odor carrying portion exits, and

the control airflow generation unit includes a second airflow generation device, and a second opening that second airflow generated by the second airflow generation device exits.

The first opening and the second opening may be provided so that movement of the first airflow exiting the first opening is able to be controlled by the second airflow.

The odor presentation module may include a first flow path through which the first airflow flows, and a second flow path through which the second airflow flows,

wherein the first flow path may be provided inside the second flow path, or may be provided outside the second flow path.

The odor carrying portion may be configured to be replaceable.

A flow path through which the airflow generated by the first airflow generation device flows may be branched into two or more flow paths, and each of the branched flow paths may have the odor carrying portion.

In the odor presentation module, an opening and closing mechanism for controlling the passage of airflow may be provided in one or both of an inlet through which the airflow generated by the first airflow generation device enters the odor carrying portion and an outlet or the first opening through which first airflow containing an odor exits the odor carrying portion.

The odor presentation module may be configured to curb the generation of airflow in the flow path through which the first airflow flows at the time of stopping of the first airflow generation device.

Further, the present technology provides an odor presentation device including: an odor presentation module including a first opening that first airflow containing an odor exits, and a second opening that second airflow exits, the first and second openings being provided so that movement of the first airflow exiting the first opening is able to be controlled by the second airflow; and

a housing configured to hold the odor presentation module.

Further, the present technology provides an odor presentation system including: an odor presentation module including a first opening that first airflow containing an odor exits, and a second opening that second airflow exits, the first and second openings being provided so that movement of the first airflow exiting the first opening is able to be controlled by the second airflow; and

a control unit configured to control the odor presentation module.

Further, the present technology provides an odor presentation method including:

a first airflow generation step of emitting first airflow containing an odor from a first opening; and

a second airflow generation step of generating second airflow exiting a second opening or second airflow entering the second opening,

wherein

movement of the first airflow exiting the first opening is controlled by the second airflow.

### BRIEF DESCRIPTION OF DRAWINGS

FIG. 1A is a schematic diagram of a configuration example of the odor presentation module according to the present technology.

FIG. 1B is a schematic diagram illustrating that an odor carrying portion can be configured as a cartridge.

FIG. 2 is a diagram illustrating a case in which unnecessary airflow is generated in a first airflow generation device according to the present technology.

FIG. 3 is a schematic diagram of a configuration example of the first airflow generation device according to the present technology.

FIG. 4A is a schematic diagram of a configuration example of the first airflow generation device according to the present technology.

FIG. 4B is a schematic diagram of a configuration example of the first airflow generation device according to the present technology.

FIG. 5A is a schematic diagram of a configuration example of the odor presentation module according to the present technology.

FIG. 5B is a schematic diagram of a configuration example of the odor presentation module according to the present technology.

FIG. 6A is a schematic diagram of a configuration example of the odor presentation module according to the present technology.

FIG. 6B is a schematic diagram of a configuration example of the odor presentation module according to the present technology.

FIG. 7 is a schematic diagram of a configuration example of the odor presentation module according to the present technology.

FIG. 8A is a schematic diagram of a configuration example of the odor presentation module according to the present technology.

FIG. 8B is a schematic diagram of a configuration example of the odor presentation module according to the present technology.

FIG. 9 is a schematic diagram of a configuration example of the odor presentation module according to the present technology.

FIG. 10 is a schematic diagram of a configuration example of the odor presentation module according to the present technology.

FIG. 11 is a schematic diagram of a configuration example of the odor presentation module according to the present technology.

FIG. 12 is a schematic diagram of a configuration example of the odor presentation module according to the present technology.

FIG. 13 is a schematic diagram of a configuration example of the odor presentation module according to the present technology.

FIG. 14 is a schematic diagram of a configuration example of an odor containing airflow generation unit included in the odor presentation module according to the present technology.

FIG. 15 is a schematic diagram of a configuration example of an odor presentation device according to the present technology.

FIG. 16 is a schematic diagram of a configuration example of the odor presentation device according to the present technology.

FIG. 17 is a diagram illustrating a configuration example of an odor presentation module included in the odor presentation device according to the present technology.

FIG. 18 is a schematic diagram of a configuration example of the odor presentation device according to the present technology.

FIG. 19 is a schematic diagram of a configuration example of the odor presentation device according to the present technology.

FIG. 20 is a schematic diagram of a configuration example of the odor presentation device according to the present technology.

FIG. 21 is a schematic diagram of a configuration example of the odor presentation device according to the present technology.

FIG. 22 is a schematic diagram of a configuration example of an odor presentation system according to the present technology.

FIG. 23 is a schematic diagram of a configuration example of the odor presentation module according to the present technology.

FIG. 24 is a schematic diagram of a configuration example of the odor presentation device according to the present technology.

FIG. 25 is a diagram illustrating the way an odor is presented using the odor presentation module according to the present technology.

### DESCRIPTION OF EMBODIMENTS

Hereinafter, preferred embodiments for carrying out the present technology will be described. The embodiments to be described hereinafter show typical embodiments of the present technology, and the scope of the present technology is not limited to these embodiments. Further, any of the embodiments can be combined. The present technology will be described in the following order.

1. First embodiment (odor presentation module)

(1) Description of first embodiment

(2) First example of first embodiment (example of odor presentation module)

(3) Second example of first embodiment (example of first airflow generation device)

(4) Third example of first embodiment (example of odor presentation module)

(5) Fourth example of first embodiment (example of odor presentation module)

(6) Fifth example of first embodiment (example of odor presentation module)

(7) Sixth example of first embodiment (example of odor presentation module)

(8) Seventh example of first embodiment (example of odor presentation module)

(9) Eighth example of first embodiment (example of odor presentation module)

(10) Ninth example of first embodiment (example of odor-containing airflow generation unit)

2. Second embodiment (odor presentation device)

3. Third embodiment (odor presentation system)

4. Fourth embodiment (odor presentation method)

1. First Embodiment (Odor Presentation Module)

(1) Description of First Embodiment

The odor presentation module according to the present technology includes a first opening that first airflow containing an odor exits and a second opening that second airflow exits, and the first opening and the second opening are provided so that movement of the first airflow exiting the first opening can be controlled by the second airflow. The odor presentation module according to the present technology can control movement of the first airflow containing the odor using the second airflow. Therefore, with the odor presentation module according to the present technology, it is possible to realize control of the odor contained in the first airflow to thereby realize various ways of presenting an odor. For example, the odor presentation module of the present technology can present an odor to a user using various expression methods such as a local odor and a drifting odor. Further, the odor presentation module of the present technology can reduce or eliminate an odor present outside the module using the second airflow.

The odor presentation module according to the present technology may be configured to be able to change the flow velocity of at least one of the first airflow and the second airflow. It is possible to realize more diverse odor expression by adjusting the flow velocity of each airflow.

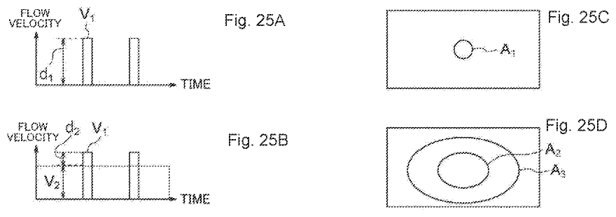

An example of the way an odor is presented using an odor presentation module according to the present technology will be described with reference to FIG. 25. FIG. 25 is a conceptual diagram illustrating an example of an odor expressed by the odor presentation module according to the present technology.

A and B of FIG. 25 illustrate a relationship between the flow velocity of the airflow reaching the user from the odor presentation module of the present technology and time. FIG. 25 is intended for conceptual description and does not show an actual flow velocity or time.

In A of FIG. 25, an example of a case in which the second airflow is not generated and the first airflow containing an odor is generated at a specific time is illustrated. In the present example, the first airflow at flow velocity V₁ reaches the user at a specific time. When the first airflow is not generated, neither the first airflow nor the second airflow is generated, and the flow velocity is 0, that is, the user does not perceive wind pressure. A difference between the flow velocity when the first airflow is not generated and the flow velocity when the first airflow is generated is d₁, as illustrated in the figure. That is, this difference d₁ is a change in wind pressure that the user perceives together with the odor.

On the other hand, B of FIG. 25 illustrates an example in which the first airflow containing an odor is generated at a specific time while the second airflow is generated. In the present example, second airflow at flow velocity V₂ is always generated. Accordingly, when the first airflow is not generated, the user always perceives wind pressure at the flow velocity V₂. The first airflow at the flow velocity V₁ is generated only when the odor presentation is necessary. Here, because the user is always receiving the wind pressure at the flow velocity V₂, a change in the wind pressure felt by the user together with the odor is a difference d₂ between V₁ and V₂.

In the case of A of FIG. 25, the user that the first airflow has reached perceives the odor and also perceives large change in wind pressure. Therefore, it is possible to express, for example, a local odor (or a suddenly generated odor) by controlling the first airflow and the second airflow as illustrated in A of FIG. 25.

On the other hand, in the case of B of FIG. 25, the user that the first airflow has reached perceives the odor, but a change in wind pressure felt by the user is smaller than in the case of A of FIG. 25. It is possible to express, for example, a drifting odor by controlling the first airflow and the second airflow as illustrated in B of FIG. 25.

Thus, the odor presentation module of the present technology enables both expression of a local odor and expression of a drifting odor.

Further, for example, it is possible to change a concentration of the odor component in the airflow reaching the user by adjusting the flow velocity V₁ of the first airflow containing the odor and/or the flow velocity V₂ of the second airflow. This makes it possible to change intensity of the odor reaching the user, for example.

The olfactory display described in PTL 1 can express a local odor as illustrated in A of FIG. 25, for example, but cannot express a drifting odor. Therefore, for example, when the olfactory display is used for odor presentation linked to a video, scenes in which the odor can be presented are limited.

As described above, the odor presentation module according to the present technology can deliver the odor to the user in various presentation methods. It is possible to express not only a local odor but also an odor drifting in a space, for example. Further, the odor present in the space can be recovered. Therefore, when the odor presentation module according to the present technology is used for odor presentation linked to a video, it is possible to realize odor expression suitable for more scenes.

Further, an aspect in which the odor presentation module according to the present technology is used is not limited to the odor presentation linked to the video. For example, the odor presentation module of the present technology can also be used for odor presentation linked to an experience (for example, an attraction in an amusement park or a play in a theater) of the user. The odor presentation module according to the present technology can be used for various types of odor expression according to content of a video or experience, and can perform, for example, spot (local) odor expression or drifting odor expression.

C and D in FIG. 25 indicate ranges that airflow exiting the odor presentation module of the present technology reaches. FIG. 25 is intended for conceptual description and does not show an actual range.

C in FIG. 25 illustrates an example in which the second airflow is not generated and only the first airflow is generated. In the present example, because only the first airflow is generated, a range that the airflow reaches is, for example, a range A₁ that the first airflow reaches.

On the other hand, D in FIG. 25 illustrates an example of a case in which the same first airflow as in the case of FIG. 25C is generated from the inside of the second airflow while the second airflow is generated. In the present example, because the second airflow and the first airflow are generated at the same time, a range that the first airflow reaches can be expanded by the second airflow. For example, when the range that the second airflow reaches is A₃, the range that the first airflow reaches is expanded by the second airflow to become an area A₂. It is possible to further adjust the range that the first airflow reaches, for example, by adjusting the flow velocity or direction of the second airflow.

Thus, with the odor presentation module according to the present technology, it is possible to adjust a range that the odor reaches.

Further, a range A₂ that the first airflow reaches in D of FIG. 25 is wider than the range A₁ in C of FIG. 25. Because the range that the first airflow containing the odor reaches is wide, it becomes difficult to specify a place from which the first airflow is emitted (that is, a position of the odor presentation module). This is useful, for example, in a case in which an odor generation source is not desired to be specified for attraction participants in an attraction such as an amusement park.

Further, because the first airflow containing the odor reaches a wider range, it is possible to present the odor to the user even in a case in which a position of a nose of the user is shifted.

As described above with reference to FIG. 25, with the odor presentation module according to the present technology, it is possible to adjust the change in wind pressure felt by the user together with the odor and/or the range that the odor reaches. This makes it possible for the odor to be delivered to the user using any of various presentation ways.

Further, the odor presentation module of the present technology can also perform control of the odor as follows using the second airflow.

For example, in a case in which the first airflow containing an odor has stayed near the first opening or near the nose of the user, it is possible to curb the staying using the second airflow. This makes it possible to reduce or eliminate the odor recognized by the user.

Further, a case in which the presentation of the odor to the user is desired to be stopped at a specific timing is assumed. In this case, only the second airflow exits the second opening from the odor presentation module of the present technology, making it possible to blow the odor component from the vicinity of the nose of the user to thereby stop the presentation of the odor. Alternatively, the second airflow entering the odor presentation module from the second opening is generated so that the odor component may be recovered into the odor presentation module. This makes it possible to reduce or eliminate the odor at a specific timing.

Thus, not only odor control from the viewpoint of presentation of an odor but also odor control from the viewpoint of reduction or elimination of an odor can be realized by the second airflow.

The odor presentation module according to the present technology may include a first airflow generation device, and an odor carrying portion that carries the odor, and may be configured such that the airflow generated by the first airflow generation device and the odor included in the odor carrying portion form the first airflow.

For example, the odor presentation module may be configured such that the first airflow is generated by the airflow generated by the first airflow generation device passing through the odor carrying portion. The first airflow exits the first opening.

Alternatively, the odor presentation module according to the present technology may be configured such that the first airflow containing an odor is formed by the airflow generated by the first airflow generation device that exits the first opening and then comes into contact with the odor carrying portion.

Further, in the odor presentation module according to the present technology, the odor carrying portion that carries the odor and the first opening may be integrated. For example, the first opening may be provided in the odor carrying portion. For example, the first opening may be provided in a cartridge described below.

Further, the odor presentation module according to the present technology may further include a second airflow generation device, and the second airflow generation device may generate the second airflow. The second airflow exits the second opening.

Thus, the first airflow and the second airflow exiting the first opening and the second opening may be generated. More specific examples of components for generating these two airflows will be described below in (2) to (10).

Further, the odor presentation module according to the present technology may include, for example, an odor-containing airflow generation unit and a control airflow generation unit that controls the odor containing airflow generated by the odor-containing airflow generation unit. The odor-containing airflow generation unit may include a first airflow generation device, an odor component carrying portion through which airflow generated by the first airflow generation device passes, and a first opening that first airflow containing an odor formed by passing through the odor component carrying portion exits. The control airflow generation unit may include a second airflow generation device, and a second opening that second airflow generated by the second airflow generation device exits.

In this embodiment, preferably, the first opening and the second opening may be provided so that movement of the first airflow exiting the first opening can be controlled by the second airflow.

In the present technology, preferably, the odor presentation module may be configured such that the flow velocity at a position 10 cm forward in a traveling direction of the first airflow from the first opening (a direction passing through a center of an opening surface of the first opening and perpendicular to the opening surface) in a case in which the control airflow generation unit is operated and the odor-containing airflow generation unit is operated can be set to equal to or smaller than twice the flow velocity at the position in a case in which the control airflow generation unit is operated and the odor-containing airflow generation unit is not operated. This makes it possible to perform the control of the first airflow using the second airflow more effectively.

In the present technology, preferably, the odor presentation module may be configured such that directivity of the odor-containing airflow generated when the odor-containing airflow generation unit and the control airflow generation unit are simultaneously operated is lower than directivity of the odor-containing airflow generated when the odor-containing airflow generation unit is operated and the control airflow generation unit is not operated. Accordingly, for example, the range reached by the first airflow is expanded by the second airflow.

(2) First Example of First Embodiment (Example of Odor Presentation Module)

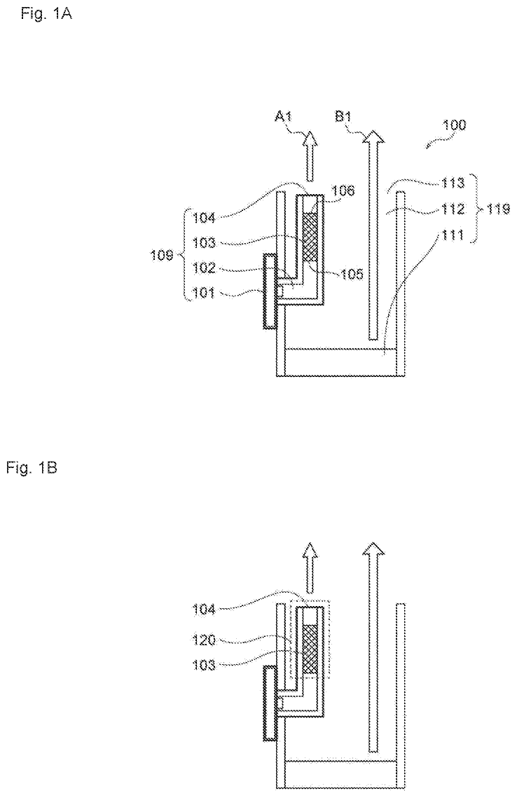

Hereinafter, an example of the odor presentation module according to the present technology will be described with reference to FIG. 1A. FIG. 1A is a schematic diagram of a configuration example of the odor presentation module according to the present technology.

As illustrated in FIG. 1A, an odor presentation module 100 includes an odor-containing airflow generation unit 109, and a control airflow generation unit 119 that controls odor-containing airflow generated by the odor-containing airflow generation unit 109.

The odor-containing airflow generation unit 109 includes a first airflow generation device 101, a flow path 102 having one end at which the first airflow generation device 101 is provided, an odor carrying portion 103 provided in the flow path 102, and a first opening 104 that is the other end of the flow path 102. These components form first airflow A1 containing an odor.

The control airflow generation unit 119 includes a second airflow generation device 111, a flow path 112 having one end to which the second airflow generation device 111 is connected, and a second opening 113 that is the other end of the flow path 112. These components form second airflow B1. The second airflow B1 controls the first airflow A1 containing an odor.

The first opening 104 is inside the second opening 113. That is, the first opening 104 and the second opening 113 are provided so that movement of the first airflow A1 exiting the first opening 104 can be controlled by the second airflow B1.

Hereinafter, these components will be described in greater detail.

The first airflow generation device 101 generates airflow flowing in the flow path 102 from the first airflow generation device 101 to the first opening 104. The first airflow generation device 101 may be, for example, an air pump, and more specifically, a diaphragm pump. The air pump may be a diaphragm pump including a piezoelectric element or a motor.

The first airflow generation device 101 is preferably configured to be able to adjust the flow velocity of the first airflow A1. For example, the first airflow generation device 101 is configured to be able to change the flow velocity of the first airflow A1 stepwise or continuously. This makes it possible to realize more types of odor expression as compared with the case of a device that generates the first airflow A1 at only one flow velocity.

The flow path 102 has the one end to which the first airflow generation device 101 is connected, and the other open end that is the first opening 104. A shape of a cross section of the flow path 102 (a cross section in a direction perpendicular to an axis of the flow path) may be, for example, a circle, an ellipse, or a rectangle (a square), and may be appropriately selected by those skilled in the art.

The odor carrying portion 103 imparts an odor to the airflow generated by the first airflow generation device 101. The odor carrying portion 103 may be provided at any position inside the flow path 102, and more preferably, the odor carrying portion 103 is provided so that the airflow generated by the first airflow generation device 101 passes through the odor carrying portion 103. For example, the odor carrying portion 103 may include an inlet 105 through which the airflow generated by the first airflow generation device 101 enters the odor carrying portion 103, and an outlet 106 through which the airflow passing through the odor carrying portion 103 exits the odor carrying portion 103. An odor can be imparted to the airflow while the airflow entering the odor carrying portion 103 from the inlet 105 passes through the odor carrying portion 103 and exits the outlet 106. More specifically, a volatile gaseous odor component may be included in the airflow. A position at which the odor carrying portion is provided may be, for example, a position closer to the first opening 104 in the flow path 102 than the first airflow generation device 101.

Further, an opening and closing mechanism or a check valve for controlling the passage of the airflow may be provided at the inlet 105 through which the airflow generated by the first airflow generation device 101 enters the odor carrying portion 103.

Further, an opening and closing mechanism or a check valve for controlling the passage of the airflow may be provided at the outlet 106 that the first airflow containing an odor exits from the odor carrying portion 103. Alternatively, an opening and closing mechanism or a check valve for controlling the passage of airflow may be provided in the first opening 104.

Thus, in the present technology, the opening and closing mechanism or the check valve for controlling the passage of the airflow may be provided in any one or both of the inlet through which the airflow generated by the first airflow generation device enters the odor carrying portion, and the outlet or the first opening through which the first airflow containing an odor exits the odor carrying portion.

It is possible to curb odor leakage from the odor carrying portion using these opening and closing mechanisms or the check valve.

The odor carrying portion 103 may include, for example, a fragrance, and may include, more specifically, a liquid fragrance or a solid fragrance. An odor component contained in a fragrance (particularly a liquid fragrance or a solid fragrance) is imparted to the airflow generated by the first airflow generation device 101, and the first airflow A1 containing the odor is formed.

The liquid fragrance may be included in the odor carrying portion 103 in a state in which the liquid fragrance is impregnated in a fragrance carrier, for example. A shape of the fragrance carrier may be, for example, a spherical shape, a square shape, a triangular shape, or a cylindrical shape. A material of the fragrance carrier may be, for example, calcium silicate, silica gel, rock wool, diatomaceous earth, zeolite, peat, charcoal, vermiculite, bentonite, pearlite, carbon nanotubes, activated charcoal, natural fiber (for example, cotton or rayon), or felt made by processing fibers.

The solid fragrance may be a solid substance obtained by solidifying a liquid fragrance using, for example, a gelling agent, or may be a solid material that emits an odor, such as a piece of wood that emits an odor.

Further, the odor carrying portion 103 may include a deodorant instead of the liquid fragrance or the solid fragrance.

The fragrance included in the odor carrying portion 103 may be, for example, a natural fragrance or a synthetic fragrance. The odor carrying portion 103 may include one fragrance or may include a combination of a plurality of fragrances.

Preferably, in the odor presentation module according to the present technology, the odor carrying portion may be configured as a cartridge. That is, the odor carrying portion may be configured to be replaceable. For example, a portion 120 of the odor presentation module indicated by a dotted line including the odor carrying portion 103 and the first opening 104 may be configured to be replaceable, as illustrated in FIG. 1B. Alternatively, only the odor carrying portion 103 may be configured to be replaceable. Alternatively, the odor carrying portion 103, the first opening 104, and a part or all of the flow path 102 may be configured to be replaceable.

The replaceable odor carrying portion makes it possible to reuse a part of the odor presentation module other than the cartridge by replacing the cartridge, for example, when the odor component included in the odor carrying portion runs out or when the user wishes to change a type of odor component included in the odor carrying portion.

The first opening 104 may be arranged inside the second opening 113, for example, as illustrated in FIG. 1A. This makes it easy to perform control using the second airflow B1 of the first airflow A1 exiting the first opening 104.

The first opening 104 is an outlet that the first airflow A1 containing an odor exits to the outside of the odor presentation module 100. A shape of an opening surface of the first opening 104 may be, for example, a circle, an ellipse, or a rectangle (square).

When the shape of the opening surface is a circle or ellipse, a diameter or long diameter may be, for example, 0.1 mm to 5 mm, particularly, 0.2 mm to 3 mm, more particularly, 0.3 mm to 2 mm, and more particularly, 0.5 mm to 1.5 mm. When the shape of the opening surface is a square or a rectangle, one side of the square or the long side of the rectangle may be, for example, 0.08 mm to 4 mm, particularly, 0.16 mm to 2.4 mm, more particularly, 0.24 mm to 1.6 mm, and more particularly, 0.4 mm to 1.2 mm.

With such sizes, it is possible to operate the first airflow generation device 101 to emit the first airflow A1 containing an odor from the first opening 104 to the outside of the odor presentation module 100 only when necessary, and to prevent the odor included in the odor carrying portion 103 from exiting the odor presentation module 100 in other cases.

A size of a cross section of the first opening 104 (particularly, a size of the shape of the opening surface of the first opening 104) may be smaller than a size of a cross section of the flow path 102 from the outlet 106 of the odor carrying portion 103 to directly before the first opening 104. For example, the size of the cross section of the first opening 104 and the size of the cross section of the flow path 102 from the outlet 106 of the odor carrying portion 103 to directly before the first opening 104 can be set so that the first airflow A1 is injected from the first opening 104. Thus, in an embodiment of the present technology, the odor presentation module (particularly, the odor-containing airflow generation unit) may be configured such that the first airflow containing an odor is injected from the first opening.

The first opening 104 may be in front of the second airflow generation device 111 in a traveling direction of the second airflow B1. For example, the opening surface of the first opening 104 may be on the same plane as an opening surface of the second opening 113, or may be in front of the plane in the traveling direction of the second airflow B1. With such a disposition, it is possible to prevent the odor component contained in the first airflow A1 from adhering to a wall surface of the flow path 112 through which the second airflow B1 flows.

The first airflow A1 emitted from the first opening 104 is airflow containing an odor, and in particular, airflow containing an odor component included in the odor carrying portion 103. For example, the first airflow A1 may be airflow of a mixture of the odor component and air in a space in which the odor presentation module is placed. The first airflow A1 emitted from the first opening 104 may be, particularly, airflow formed by injection from the first opening 104, and may be, for example, directional airflow.

As illustrated in FIG. 1A, the flow path 102 including the odor carrying portion 103 may be provided inside the flow path 112 having the one end at which the second airflow generation device 111 is provided. This makes it easy to perform control of the first airflow A1 using the second airflow B1. This also makes it easy to perform the presentation of the drifting odor.

The second airflow generation device 111 forms airflow that flows in the flow path 112 in a direction from the second airflow generation device 111 to the second opening 113. The second airflow generation device 111 may form the second airflow B1 exiting the second opening 113.

The second airflow generation device 111 may be configured to also be able to form airflow flowing in an opposite direction. That is, the second airflow generation device 111 may be configured to be able to reverse a direction of airflow that is generated. Thus, the odor presentation module according to the present technology may be configured to change a direction of the second airflow to a direction into the second opening. This makes it possible to perform recovery of the odor component and to perform odor control such as reducing or eliminating the odor.

The second airflow generation device 111 may be, for example, a blowing device. More specifically, examples of the blowing device may include an axial fan, a blower fan, and a centrifugal fan, and particularly, the blowing device is an axial fan. For example, the odor presentation module 1500 according to the present technology may include an axial fan 1501 as the second airflow generation device, as illustrated in A of FIG. 23.

Alternatively, the blowing device may be a combination of a diaphragm pump, particularly, a diaphragm pump using a piezoelectric element or a motor (for example, a microblower) and an airflow expansion mechanism. The blowing device may be configured such that the airflow generated by the diaphragm pump is expanded by the airflow expansion mechanism and flows through the flow path. For example, the odor presentation module 1550 according to the present technology may be a combination of a diaphragm pump 1502 and an airflow expansion mechanism 1503 as the second airflow generation device, as illustrated in B of FIG. 23.

Each of the odor presentation modules 1500 and 1550 illustrated in FIG. 23 includes two odor-containing airflow generation units 1504. Thus, the odor presentation module according to the present technology may include one odor-containing airflow generation unit, or may include a plurality of (for example, 2 to 10, and particularly, 2 to 6) odor-containing airflow generation units.

The second airflow B1 generated by the second airflow generation device 111 may be airflow including the air itself in the space in which the odor presentation module 100 is placed, or may be airflow including air obtained by any other component being mixed with the air in the space. Preferably, the second airflow B1 is the air itself in the space in which the odor presentation module 100 is placed. This makes it easier to perform control of the presentation of the odor of the first airflow A1. For example, the second airflow B1 may be odorless air.

The second airflow generation device 111 is preferably configured to be able to adjust the flow velocity of the second airflow B1. For example, the second airflow generation device 111 is configured to be able to change the flow velocity of the second airflow B1 stepwise or continuously. This makes it possible to realize more types of odor expression as compared with the case of a device that generates the second airflow B1 at only one flow velocity.

The flow path 112 has one end to which the second airflow generation device 111 is connected, and the other end that is open and is the second opening 113. A shape of a cross section of the flow path 112 (a cross section in a direction perpendicular to an axis of the flow path) may be, for example, a circle, an ellipse, or a rectangle (a square), and may be appropriately selected by those skilled in the art. A size of the cross section of the flow path 112 through which the second airflow B1 flows is preferably larger than the size of the cross section of the flow path 102 through which the first airflow A1 flows.

The flow path 102 through which the first airflow A1 flows is provided inside the flow path 112 through which the second airflow B1 flows. Thus, the odor presentation module according to the present technology includes a first flow path through which the first airflow flows, and a second flow path through which the second airflow flows, and the first flow path may be provided inside the second flow path.

The second opening 113 is an opening that the second airflow B1 formed using the second airflow generation device 111 exits. An area of the opening surface of the second opening 113 is preferably larger than an area of the opening surface of the first opening 104. For example, the area of the opening surface of the second opening 113 may be 3 to 1000 times, particularly 5 to 500 times, and more particularly, 10 to 200 times the area of the opening surface of the first opening 104. This makes it easier to perform control of the first airflow using the second airflow.

A shape of the opening surface of the second opening 113 may be, for example, a circle, an ellipse, or a rectangle (square). When the shape of the opening surface is a circle or ellipse, a diameter or long diameter may be, for example, 5 mm to 1000 mm, particularly 7 mm to 500 mm, and more particularly 10 mm to 100 mm. When the shape of the opening surface is a square or rectangle, one side of the square or a long side of the rectangle may be, for example, 5 mm to 1000 mm, particularly 7 mm to 500 mm, and more particularly 10 mm to 100 mm.

As illustrated in FIG. 1A, the second airflow generation device 111 may be disposed behind the first opening 104 that the first airflow A1 containing an odor exits (behind a position on an axis of the first opening 104 when a traveling direction of the first airflow A1 is the axis). Alternatively, the second airflow generation device 111 may be located in front of the first opening 104.

Preferably, as illustrated in FIG. 1A, the second airflow generation device 111 is disposed behind the first opening 104 that the first airflow A1 containing an odor exits. This makes it possible to prevent the odor component contained in the first airflow A1 containing an odor from adhering to the second airflow generation device 111 in a case in which the second airflow generation device 111 emits the second airflow B1 from the second opening 113.

In the odor presentation module 100 described above, the movement of the first airflow A1 exiting the first opening 104 can be controlled by the second airflow B1

For example, it is possible to generate the first airflow A1 while continuously generating the second airflow B1 exiting the second opening 113, to thereby present, for example, the drifting odor to the user. It is possible to increase or decrease a flow rate (air volume) of the second airflow B1, to thereby change a degree of odor of the first airflow A1. Further, it is possible to stop the generation of the second airflow B1, to thereby generate a local odor due to the first airflow A1.

Further, it is possible to generate the second airflow B1 entering the odor presentation module from the second opening 113, to thereby recover the first airflow A1 containing an odor. For example, it is possible to recover the odor emitted to the outside of the odor presentation module. This makes it possible to decrease a degree of the odor recognized by the user, or to prevent the user from recognizing the odor.

For the odor presentation module 100 illustrated in FIG. 1, the first opening 104 and the second opening 113 may be provided on an outer surface of the odor presentation module 100. This makes it possible for the first airflow A1 and the second airflow B1 to come into contact with each other outside the odor presentation module 100. Thus, the odor presentation module of the present technology may be configured such that the first airflow and the second airflow come into contact with each other outside the odor presentation module.

As described above, the odor presentation module according to the present technology may include, for example, the odor-containing airflow generation unit, and the control airflow generation unit that controls the odor-containing airflow generated by the odor-containing airflow generation unit. The odor-containing airflow generation unit may include a first airflow generation device, an odor component carrying portion through which airflow generated by the first airflow generation device passes, and a first opening that first airflow containing an odor formed by passing through the odor component carrying portion exits. The control airflow generation unit may include a second airflow generation device, and a second opening that second airflow generated by the second airflow generation device exits.

(3) Second Example of First Embodiment (Example of First Airflow Generation Device)

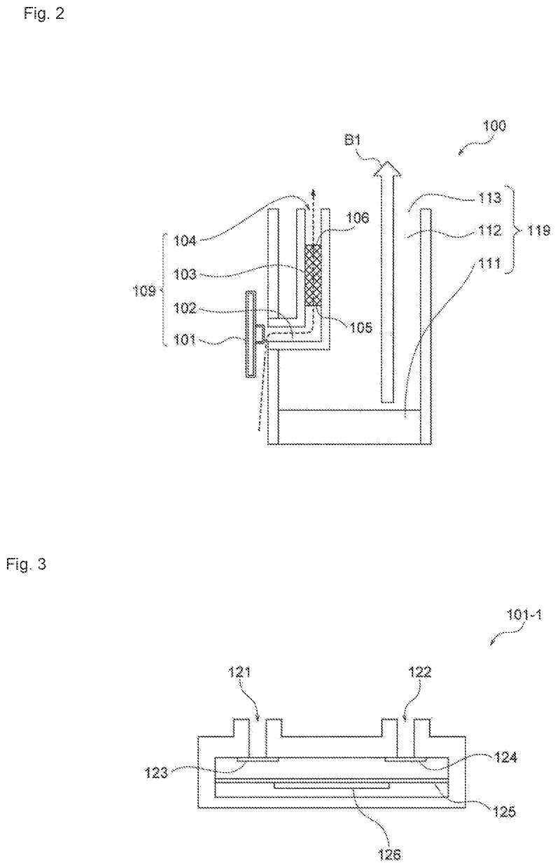

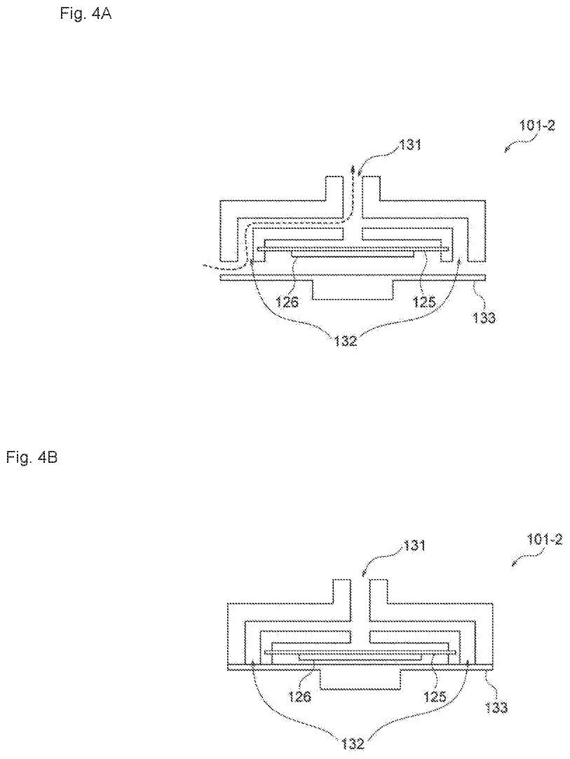

Another example of the first airflow generation device according to the present technology will be described with reference to FIGS. 2 to 4. FIG. 2 is a diagram illustrating a case in which an unnecessary airflow is generated in the first airflow generation device according to the present technology. FIG. 3 is a schematic diagram of the first airflow generation device of the other example.

In order to form a state in which the user perceives only airflow without perceiving an odor, it is conceivable to operate the second airflow generation device 111 without operating the first airflow generation device 101. In order to form the above state, it is required to reliably prevent the odor from exiting the module from the first opening 104. However, with the generation of the second airflow B1 using the second airflow generation device 111, the odor may exit the module from the first opening 104 even when the first airflow generation device 101 is not operated. It is considered that this is caused, for example, by a gap being present in a portion in which the first airflow generation device 101 and the flow path 102 are connected, or by the first airflow generation device 101 having a structure in which a gap from the outside of the module to the inside of the flow path 102 is included. That is, due to the presence of such a gap, the generation of the second airflow B1 using the second airflow generation device 111 can induce generation of airflow in the flow path 102 at the time of stopping of an operation of the first airflow generation device 101.

For example, as illustrated in FIG. 2, when the odor presentation module 100 is actually used, in a case in which there is a gap in a portion in which the first airflow generation device 101 and the flow path 102 of the odor-containing airflow generation unit 109 are connected, the second airflow generation device 111 is operated even when the first airflow generation device 101 is not operated, so that airflow (indicated by a dotted line in FIG. 2) is generated in the flow path 102 and an odor is emitted at an unintended timing. Further, in FIG. 2, the first airflow generation device 101 is shown to be separated from the flow path 102, and this is intended to emphasize and show the presence of the gap, and does not show a structure in an actual device.

The emission of the odor at the unintended timing as described above is not desirable, and may lead to a loss of the fragrance included in the odor carrying portion 103.

Further, the same problem as described above can occur in a case in which the opening 104 of the flow path 102 is provided in an open structure in the flow path 112 of the control airflow generation unit 119 even when there is no gap in the flow path 102.

Therefore, in a second example of the present embodiment, a configuration example of the first airflow generation device that curbs generation of airflow in the flow path 102 of the odor-containing airflow generation unit 109 when the second airflow generation device 111 is operated at a time when the operation of the first airflow generation device stops is shown.

As illustrated in FIG. 3, a first airflow generation device 101-1 in the odor-containing airflow generation unit 109 includes a discharge port 121 formed on the side communicating with the flow path 102 and discharging the first airflow, and an intake port 122 for taking in the first airflow.

The discharge port 121 and the intake port 122 communicate with an internal space of the first airflow generation device 101-1, and the flow path 102. An opening and closing valve 123 that blocks the discharge port 121, and an opening and closing valve 124 that blocks the intake port 122 are provided inside the first airflow generation device 101-1.

Further, a diaphragm 125 at positions facing the opening and closing valve 123 and the opening and closing valve 124, and a piezoelectric element 126 connected to the diaphragm 125 are provided inside the first airflow generation device 101-1. By the diaphragm 125 having the piezoelectric element 126 operated as a pump, first airflow is generated and the opening and closing valve 123 and the opening and closing valve 124 are opened or closed.

The odor presentation module 100 using the first airflow generation device 101-1 for the odor-containing airflow generation unit 109 has a structure in which there is no gap in the flow path 102 of the odor-containing airflow generation unit 109, and closes the opening and closing valve 123 and the opening and closing valve 124 to block the discharge port 121 and the intake port 122 at the time of stopping of an operation of the first airflow generation device 101-1.

Therefore, the odor presentation module 100 of the present example can curb generation of airflow in the flow path 102 even when the second airflow generation device 111 is operated at the time of stopping of the operation of the first airflow generation device 101-1. This makes it possible for the odor presentation module 100 of the present example to curb the emission of the odor from the flow path 102 at an unintended timing or a loss of a fragrance included in the odor carrying portion 103.

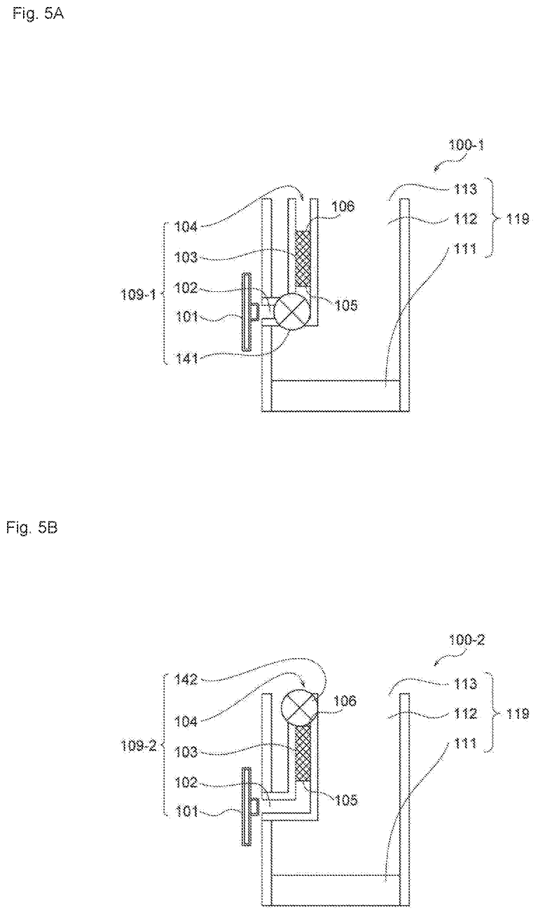

Next, a first airflow generation device according to the modification example of the present example will be described with reference to FIG. 4. FIG. 4 is a schematic diagram of the first airflow generation device of the modification example. FIG. 4A illustrates a case in which an operation of the first airflow generation device is ON. FIG. 4B illustrates a case in which the operation of the first airflow generation device is OFF.

As illustrated in FIGS. 4A and 4B, the first airflow generation device 101-2 in the odor-containing airflow generation unit 109 includes a discharge port 131 for discharging the first airflow, which is formed on the side communicating with the flow path 102, and two intake ports 132 for taking in the first airflow, which is formed on side opposite to a side communicating with the flow path 102. The numbers of the discharge port 131 and the intake port 132 may be one or plural. Further, the discharge port 131 and the intake port 132 may be disposed at positions facing each other or at other positions.

The discharge port 131 communicates an internal space of the first airflow generation device 101-2 and the flow path 102. Further, the discharge port 131 and the two intake ports 132 communicate with each other in the internal space of the first airflow generation device 101-2. Further, a diaphragm 125 extending in a direction substantially orthogonal to a direction in which the discharge port 131 and the intake port 132 face each other, and a piezoelectric element 126 connected to the diaphragm 125 are provided inside the first airflow generation device 101-2.

An opening and closing valve 133 that blocks the two intake ports 132 is provided on the side in which the intake ports 132 of the first airflow generation device 101-2 are formed. As illustrated in FIG. 4A, when an operation of the first airflow generation device 101-2 is ON, the opening and closing valve 133 is opened to separate from the two intake ports 132, and the first airflow flows from the intake port 132 to the discharge port 131. On the other hand, as illustrated in FIG. 4B, when the operation of the first airflow generation device 101-2 is OFF, the opening and closing valve 133 is closed to block the two intake ports 132, and the first airflow does not flow from the intake port 132 to the discharge port 131.

The odor presentation module 100 using the first airflow generation device 101-2 for the odor-containing airflow generation unit 109 has a structure in which there is no gap in the flow path 102 of the odor-containing airflow generation unit 109, and closes the opening and closing valve 133 to block the two intake ports 132 at the time of stopping of the operation of the first airflow generation device 101-2.

Therefore, the odor presentation module 100 of the modification example of the present example can curb generation of airflow in the flow path 102 even when the second airflow generation device 111 is operated at the time of stopping of the operation of the first airflow generation device 101-2. This makes it possible for the odor presentation module 100 of the modification example of the present example to curb the emission of the odor from the flow path 102 at an unintended timing or a loss of a fragrance included in the odor carrying portion 103.

(4) Third Example of First Embodiment (Example of Odor Presentation Module)

Another example of the odor presentation module according to the present technology will be described with reference to FIGS. 5 to 7. FIGS. 5A and 5B are schematic views of the odor presentation module of the other example, respectively.

A third example of the present embodiment shows a configuration example of the odor presentation module that curbs generation of the airflow in the flow path of the odor-containing airflow generation unit when the second airflow generation device 111 is operated at a time when the operation of the first airflow generation device stops.

As illustrated in FIG. 5A, the odor presentation module 100-1 includes an odor-containing airflow generation unit 109-1, and a control airflow generation unit 119 that controls an odor-containing airflow generated by the odor-containing airflow generation unit 109-1, as in the first example.

The odor-containing airflow generation unit 109-1 includes the first airflow generation device 101, the flow path 102 having one end at which the first airflow generation device 101 is provided, the odor carrying portion 103 provided in the flow path 102, and the first opening 104 that is the other end of the flow path 102. These components form the first airflow containing an odors.

Further, in the flow path 102, a solenoid valve 141 that is an example of an opening and closing valve is provided between the first airflow generation device 101 and the odor carrying portion 103. The solenoid valve 141 can be electrically controlled so that solenoid valve 141 is opened or closed, and blocks the flow path 102 at the time of stopping of the operation of the first airflow generation device 101.

As illustrated in FIG. 5B, the odor presentation module 100-2 includes an odor-containing airflow generation unit 109-2, and a control airflow generation unit 119 that controls the odor-containing airflow generated by the odor-containing airflow generation unit 109-2, similar to FIG. 5A.

The odor-containing airflow generation unit 109-2 includes the first airflow generation device 101, the flow path 102 having one end at which the first airflow generation device 101 is provided, the odor carrying portion 103 provided in the flow path 102, and the first opening 104 that is the other end of the flow path 102.

Further, a solenoid valve 142 is provided between the odor carrying portion 103 and the first opening 104 in the flow path 102. The solenoid valve 142 blocks the flow path 102 at the time of stopping of the operation of the first airflow generation device 101, similar to the solenoid valve 141. The solenoid valve is not limited to the disposition in the present example, and may be disposed at any position in the flow path 102 as long as the flow path 102 can be blocked at the time of stopping of the operation of the first airflow generation device 101.

The first airflow generation device 101, the flow path 102, the odor carrying portion 103, and the first opening 104 included in the odor-containing airflow generation unit 109-1 and the odor-containing airflow generation unit 109-2 may be the same as the respective components included in the odor-containing airflow generation unit 109 described in (2) above, and the description thereof also applies to the present example. Further, the respective components included in the control airflow generation unit 119 may be the same as the respective components included in the control airflow generation unit 119 described in (2) above, and the description thereof also applies to the present example.

The odor presentation module 100-1 including the odor-containing airflow generation unit 109-1 or the odor presentation module 100-2 including the odor-containing airflow generation unit 109-2 closes the solenoid valve 141 or the solenoid valve 142 to block the flow path 102 at the time of stopping of the operation of the first airflow generation device 101.

Therefore, in the odor presentation module 100-1 and the odor presentation module 100-2, even when there is a gap, for example, between the first airflow generation device 101 and the flow path 102, it is possible to curb generation of airflow in the flow path 102 when the second airflow generation device 111 is operated at the time of stopping of an operation of the first airflow generation device 101. This makes it possible for the odor presentation module 100-1 and the odor presentation module 100-2 to curb the emission of the odor from the flow path 102 at an unintended timing or a loss of a fragrance included in the odor carrying portion 103.

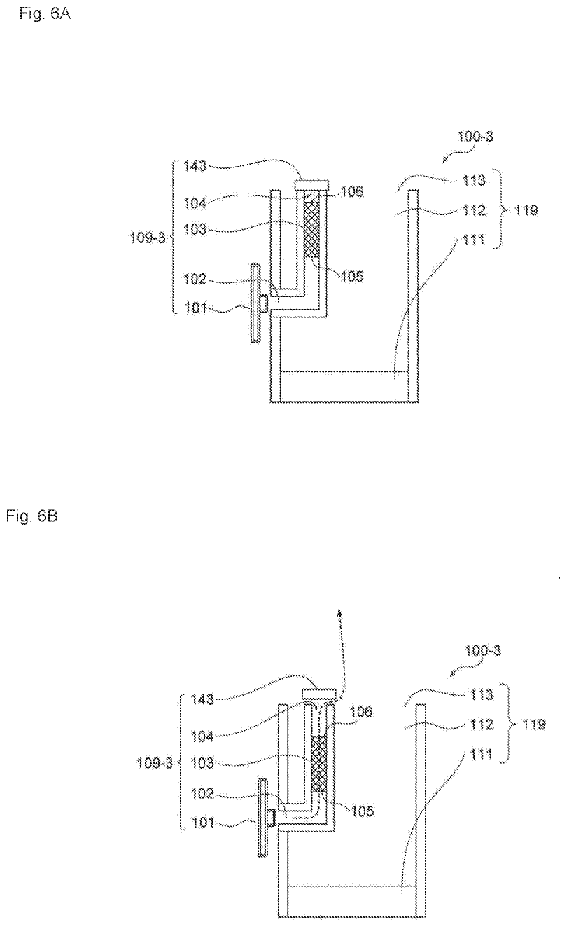

Next, an odor presentation module according to a modification example of the present example will be described with reference to FIG. 6. FIG. 6 is a schematic diagram of the odor presentation module of the modification example. FIG. 6A illustrates a case in which the operation of the first airflow generation device is OFF. FIG. 6B illustrates a case in which the operation of the first airflow generation device is ON.

As illustrated in FIGS. 6A and 6B, an odor presentation module 100-3 includes an odor-containing airflow generation unit 109-3, and a control airflow generation unit 119 that controls an odor-containing airflow generated by the odor-containing airflow generation unit 109-3, as in the first example.

The odor-containing airflow generation unit 109-3 includes the first airflow generation device 101, the flow path 102 having one end at which the first airflow generation device 101 is provided, the odor carrying portion 103 provided in the flow path 102, and the first opening 104 that is the other end of the flow path 102.

Further, the first opening 104 is provided with an opening and closing valve 143 that opens at a specific pressure. As illustrated in FIG. 6A, when the operation of the first airflow generation device 101 is OFF, the opening and closing valve 143 closes the first opening 104 to block the flow path 102, and no airflow flows through the flow path 102. On the other hand, as illustrated in FIG. 6B, when the operation of the first airflow generation device 101 is ON, the opening and closing valve 143 opens and separates from the first opening 104 so that the airflow flows in the flow path 102. Thus, the opening and closing valve 143 blocks the flow path 102 by closing the first opening 104 at the time of stopping of an operation of the first airflow generation device 101.

The first airflow generation device 101, the flow path 102, the odor carrying portion 103, and the first opening 104 included in the odor-containing airflow generation unit 109-3 may be the same as the respective components included in the odor-containing airflow generation unit 109 described in (2) above, and the description thereof also applies to the present example. Further, the respective components included in the control airflow generation unit 119 may be the same as the respective components included in the control airflow generation unit 119 described in (2) above, and the description thereof also applies to the present example.

The odor presentation module 100-3 including the odor-containing airflow generation unit 109-3 closes the opening and closing valve 143 to block the flow path 102 at the time of stopping of the operation of the first airflow generation device 101.

Therefore, in the odor presentation module 100-3, even when there is a gap, for example, between the first airflow generation device 101 and the flow path 102, it is possible to curb generation of airflow in the flow path 102 when the second airflow generation device 111 is operated at the time of stopping of an operation of the first airflow generation device 101. This makes it possible for the odor presentation module 100-3 to curb the emission of the odor from the flow path 102 at an unintended timing or a loss of a fragrance included in the odor carrying portion 103.

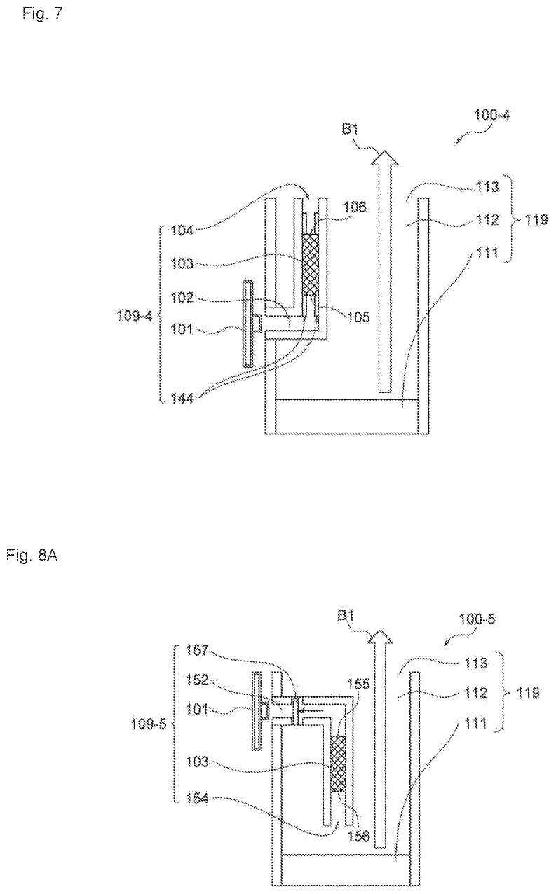

Next, an odor presentation module according to another modification example of the present example will be described with reference to FIG. 7. FIG. 7 is a schematic diagram of the odor presentation module of the other modification example.

As illustrated in FIG. 7, the odor presentation module 100-4 includes an odor-containing airflow generation unit 109-4, and a control airflow generation unit 119 that controls an odor-containing airflow generated by the odor-containing airflow generation unit 109-4, as in the first example.

The odor-containing airflow generation unit 109-4 includes the first airflow generation device 101, the flow path 102 having one end at which the first airflow generation device 101 is provided, the odor carrying portion 103 provided in the flow path 102, and the first opening 104 that is the other end of the flow path 102.

Further, a shield 144 is provided in the flow path 102 near the inlet 105 and the outlet 106 of the odor carrying portion 103. The shield 144 narrows the inside of the flow path 102, and shields a flow of airflow generated in the path 102 at the time of stopping of the operation of the first airflow generation device 101, or reduces a flow rate of the airflow. The shield 144 may be provided at any one of the inlet 105 and the outlet 106 of the odor carrying portion 103.

The first airflow generation device 101, the flow path 102, the odor carrying portion 103, and the first opening 104 included in the odor-containing airflow generation unit 109-4 may be the same as the respective components included in the odor-containing airflow generation unit 109 described in (2) above, and the description thereof also applies to the present example. Further, the respective components included in the control airflow generation unit 119 may be the same as the respective components included in the control airflow generation unit 119 described in (2) above, and the description thereof also applies to the present example.

The odor presentation module 100-4 including the odor-containing airflow generation unit 109-4 narrows the inside of the flow path 102 using the shield 144 at the time of stopping of the operation of the first airflow generation device 101, thereby reducing a flow rate of the airflow generated in the path 102 at the time of stopping of the operation of the first airflow generation device 101.

Therefore, in the odor presentation module 100-4, even when there is a gap, for example, between the first airflow generation device 101 and the flow path 102, it is possible to curb generation of airflow in the flow path 102 when the second airflow generation device 111 is operated at the time of stopping of an operation of the first airflow generation device 101. This makes it possible for the odor presentation module 100-4 to curb the emission of the odor from the flow path 102 at an unintended timing or a loss of a fragrance included in the odor carrying portion 103.

(5) Fourth Example of First Embodiment (Example of Odor Presentation Module)

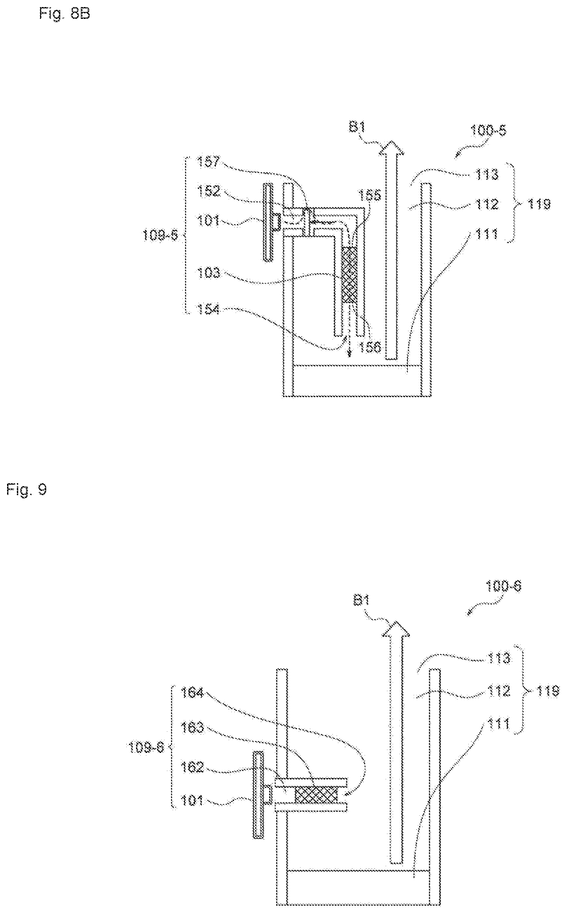

Another example of the odor presentation module according to the present technology will be described with reference to FIG. 8. FIG. 8 is a schematic diagram of the odor presentation module of the other example. FIG. 8A illustrates a case in which the operation of the first airflow generation device is OFF. FIG. 8B illustrates a case in which the operation of the first airflow generation device is ON.

As illustrated in FIGS. 8A and 8B, the odor presentation module 100-5 includes an odor-containing airflow generation unit 109-5, and the control airflow generation unit 119 that controls the odor-containing airflow generated by the odor-containing airflow generation unit 109-5.

The odor-containing airflow generation unit 109-5 includes a first airflow generation device 101, a flow path 152 having one end at which the first airflow generation device 101 is provided, an odor carrying portion 103 provided in the flow path 152, and a first opening 154 that is the other end of the flow path 152. These components form first airflow containing an odor.

The first opening 154 opens to face a direction in which the second airflow B1 blown out from the second airflow generation device 111 flows, inside the flow path 112 of the control airflow generation unit 119. That is, the first opening 154 is provided so that movement of the first airflow exiting the first opening 154 can be controlled by the second airflow B1. The second airflow B1 may be a discharge airflow or may be an intake airflow.

Further, in the flow path 152, a check valve 157 that is an example of an opening and closing valve is provided between the first airflow generation device 101 and the odor carrying portion 103. The check valve 157 is opened when the first airflow flows from the first airflow generation device 101 to the first opening 154 through the flow path 152, and is closed when the airflow flows from the first opening 154 to the first airflow generation device 101 through the flow path 152, to block the flow path 152.

The first airflow generation device 101, the flow path 152, the odor carrying portion 103, and the first opening 154 included in the odor-containing airflow generation unit 109-5 may be the same as the respective components included in the odor-containing airflow generation unit 109 described in (2) above, and the description thereof also applies to the present example. Further, the respective components included in the control airflow generation unit 119 may be the same as the respective components included in the control airflow generation unit 119 described in (2) above, and the description thereof also applies to the present example. In FIGS. 8A and 8B, an upper side of the odor carrying portion 103 is an inlet 155 through which the airflow generated by the first airflow generation device 101 enters the odor carrying portion 103, and a lower side of the odor carrying portion 103 is an outlet 166 through which the airflow passing through the odor carrying portion 103 exits the odor carrying portion 103.

The odor presentation module 100-5 including the odor-containing airflow generation unit 109-5 closes the check valve 157 to block the flow path 152 when the airflow flows back in the flow path 152 from the first opening 154 to the first airflow generation device 101.

Therefore, it is possible for the odor presentation module 100-5 to curb the generation of airflow flowing back through the flow path 152 when there is a gap in the flow path 152 or when the second airflow generation device 111 is operated at the time of stopping of the operation of the first airflow generation device 101. This makes it possible for the odor presentation module 100-5 to curb the emission of an odor from the flow path 152 at an unintended timing or a loss of the fragrance included in the odor carrying portion 103.

(6) Fifth Example of First Embodiment (Example of Odor Presentation Module)

Another example of the odor presentation module according to the present technology will be described with reference to FIG. 9. FIG. 9 is a schematic diagram of the odor presentation module of the other example.

As illustrated in FIG. 9, the odor presentation module 100-5 includes an odor-containing airflow generation unit 109-6, and a control airflow generation unit 119 that controls odor-containing airflow generated by the odor-containing airflow generation unit 109-6.

The odor-containing airflow generation unit 109-6 includes a first airflow generation device 101, a flow path 162 having one end at which the first airflow generation device 101 is provided, an odor carrying portion 163 provided in the flow path 162, and a first opening 164 that is the other end of the flow path 162. These components form the first airflow containing an odors.

The first opening 164 opens in a direction perpendicular to a direction in which the second airflow B1 blown out from the second airflow generation device 111 flows, inside the flow path 112 of the control airflow generation unit 119. That is, the first opening 164 is provided so that movement of the first airflow exiting the first opening 164 can be controlled by the second airflow B1.

The first airflow generation device 101, the flow path 162, the odor carrying portion 163, and the first opening 164 included in the odor-containing airflow generation unit 109-6 may be the same as the respective components included in the odor-containing airflow generation unit 109 described in (2) above, and the description thereof also applies to the present example. Further, the respective components included in the control airflow generation unit 119 may be the same as the respective components included in the control airflow generation unit 119 described in (2) above, and the description thereof also applies to the present example.

Because the odor presentation module 100-6 including the odor-containing airflow generation unit 109-6 is open in the direction perpendicular to the direction in which the second airflow B1 flows, it is possible to curb the generation of airflow in the flow path 162 when there is a gap in the flow path 162, or when the second airflow generation device 111 is operated at the time of stopping of an operation of the first airflow generation device 101. This makes it possible for the odor presentation module 100-6 can curb the emission of an odor from the flow path 162 at an unintended timing or a loss of the fragrance included in the odor carrying portion 163.

(7) Sixth Example of First Embodiment (Example of Odor Presentation Module)

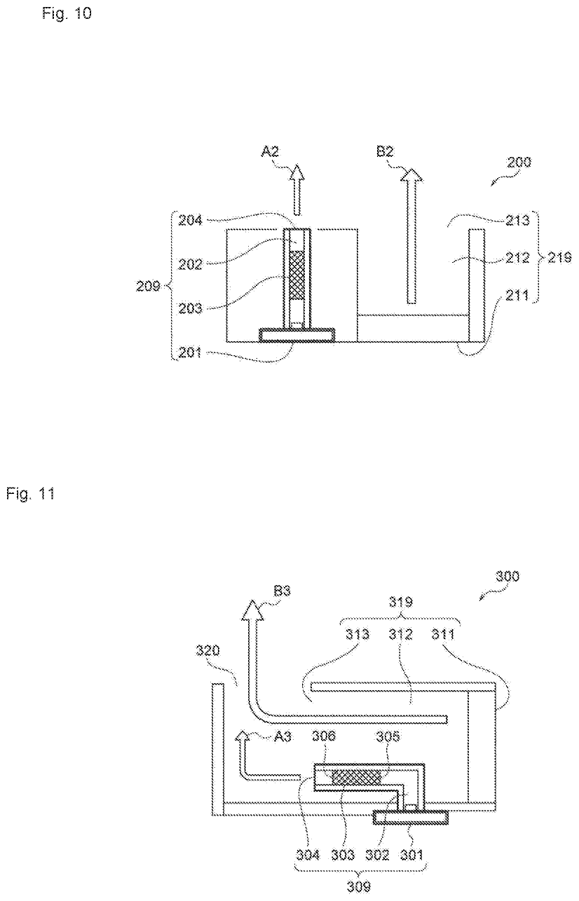

Another example of the odor presentation module according to the present technology will be described with reference to FIG. 10. FIG. 10 is a schematic diagram of the odor presentation module of the other example.

As illustrated in FIG. 10, the odor presentation module 200 includes an odor-containing airflow generation unit 209, and a control airflow generation unit 219 that controls an odor-containing airflow generated by the odor-containing airflow generation unit 209.

The odor-containing airflow generation unit 209 includes a first airflow generation device 201, a flow path 202 having one end at which the first airflow generation device 201 is provided, an odor carrying portion 203 provided in the flow path 202, and a first opening 204 that is the other end of the flow path 202. These components form first airflow A2 containing an odor.

The control airflow generation unit 219 includes a second airflow generation device 211, a flow path 212 having an one end at which the second airflow generation device 211 is provided, and a second opening 213 that is the other end of the flow path 212. These components form second airflow B2. The second airflow B2 controls the first airflow A2 containing an odor.

The first opening 204 is near the second opening 213. That is, the first opening 204 and the second opening 213 are provided so that movement of the first airflow A2 exiting the first opening 204 can be controlled by the second airflow B2.

The odor-containing airflow generation unit 209, and the first airflow generation device 201, the flow path 202, the odor carrying portion 203, and the first opening 204 included in the odor-containing airflow generation unit 209 may be the same as the odor-containing airflow generation unit 109 described in (2) above, and the first airflow generation device 101, the flow path 102, the odor carrying portion 103, and the first opening 104 included therein, and the description thereof also applies to the present example.

The control airflow generation unit 219, and the second airflow generation device 211, the flow path 212, and the second opening 213 included therein may be the same as the control airflow generation unit 119 described in (2) above, and the second airflow generation device 111, the flow path 112, and the second opening 113 included therein, and the description thereof also applies to the present example.

As illustrated in FIG. 10, the first opening 204 is outside the second opening 213. Thus, in the odor presentation module 200 according to the present technology, the first opening may be outside the second opening. According to the odor presentation module 200, it is possible to curb emission of an odor from the flow path 202 at an unintended timing or a loss of a fragrance included in the odor carrying portion 203, similar to the odor presentation module.

Further, as illustrated in FIG. 10, the flow path 202 through which the first airflow A2 flows is provided outside the flow path 212 through which the second airflow B2 flows. Thus, the odor presentation module according to the present technology may include a first flow path through which the first airflow flows, and a second flow path through which the second airflow flows, and the first flow path may be provided outside the second flow path.

The control of the odor as described in (2) above can be performed by the odor presentation module 200 illustrated in FIG. 10. In this case, the first opening 204 and the second opening 213 may be configured such that the first airflow A2 and the second airflow B2 come into contact with each other outside the odor presentation module. For example, the first opening 204 may be disposed near the second opening 213 so that the first airflow A2 and the second airflow B2 come into contact with each other outside the odor presentation module.

(8) Seventh example of First Embodiment (Example of Odor Presentation Module)

Another example of the odor presentation module according to the present technology will be described with reference to FIG. 11. FIG. 11 is a schematic diagram of the odor presentation module of the other example.

As illustrated in FIG. 11, the odor presentation module 300 includes an odor-containing airflow generation unit 309, and a control airflow generation unit 319 that controls an odor-containing airflow generated by the odor-containing airflow generation unit 309.

The odor-containing airflow generation unit 309 includes a first airflow generation device 301, a flow path 302 having one end at which the first airflow generation device 301 is provided, an odor carrying portion 303 provided in the flow path 302, and a first opening 304 that is the other end of the flow path 302. These components form first airflow A3 containing an odor.

The control airflow generation unit 319 includes a second airflow generation device 311, a flow path 312 having a one end at which the second airflow generation device 311 is provided, and a second opening 313 that is the other end of the flow path 312. These components form second airflow B3. The second airflow B3 controls the first airflow A3 containing an odor.

The first opening 304 is inside the second opening 313. That is, the first opening 304 and the second opening 313 are provided so that movement of the first airflow A3 exiting the first opening 304 can be controlled by the second airflow B3.

Further, the first opening 304 and the second opening 313 are provided inside the odor presentation module 300. Accordingly, the first airflow A3 and the second airflow B3 come into contact with each other inside the odor presentation module 300. The odor presentation module 300 includes an opening 320 through which airflow formed by these airflows coming into contact with each other exits.

The odor-containing airflow generation unit 309, and the first airflow generation device 301, the flow path 302, the odor carrying portion 303, and the first opening 304 included in the odor-containing airflow generation unit 309 may be the same as the odor-containing airflow generation unit 109 described in (2) above, and the first airflow generation device 101, the flow path 102, the odor carrying portion 103, and the first opening 104 included therein, and the description thereof also applies to the present example.

The control airflow generation unit 319, and the second airflow generation device 311, the flow path 312, and the second opening 313 included therein may be the same as the control airflow generation unit 119 described in (2) above, and the second airflow generation device 111, the flow path 112, and the second opening 113 included therein, and the description thereof also applies to the present example.

As illustrated in FIG. 11, the first opening 304 and the second opening 313 may be provided inside the odor presentation module 300. The control of the odor as described in (2) above can be performed by the odor presentation module 300 illustrated in FIG. 11.

The odor presentation module 300 illustrated in FIG. 11 differs from the odor presentation module 100 illustrated in FIG. 1, and the first opening 304 and the second opening 313 are provided inside the odor presentation module 300. That is, the first airflow A3 and the second airflow B3 come into contact with each other inside the odor presentation module 300. Thus, the odor presentation module of the present technology may be configured such that the first airflow and the second airflow come into contact with each other inside the odor presentation module.

(9) Eighth Example of First Embodiment (Example of Odor Presentation Module)

The odor presentation module according to the present technology may include one odor-containing airflow generation unit and one control airflow generation unit, may include a plurality of odor-containing airflow generation units and one control airflow generation unit, may include one odor-containing airflow generation unit and a plurality of control airflow generation units, or may include a plurality of odor-containing airflow generation units and a plurality of control airflow generation units. The odor presentation module described in (2) to (8) above includes one odor containing airflow generation unit and one control airflow generation unit.

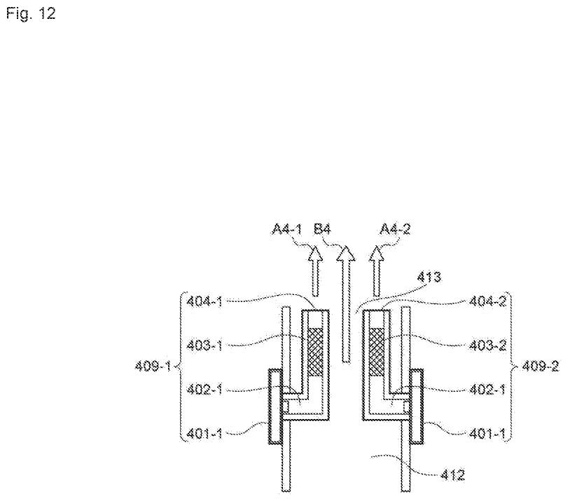

Hereinafter, a configuration example in a case in which a plurality of odor-containing airflow generation units and one control airflow generation unit are included will be described with reference to FIG. 12.

The odor presentation module 400 illustrated in FIG. 12 includes two odor-containing airflow generation units 409-1 and 409-2, and a control airflow generation unit 419 that controls odor-containing airflow generated by these odor containing airflow generation units.

The odor-containing airflow generation unit 409-1 includes a first airflow generation device 401-1, a flow path 402-1 having one end at which the first airflow generation device 401-1 is provided, an odor carrying portion 403-1 provided in the flow path 402-1, and a first opening 404-1 that is the other end of the flow path 402-1. These components form first airflow A4-1 containing an odor. The odor-containing airflow generation unit 409-2 also includes the same components and forms first airflow A4-2 containing an odor.

The control airflow generation unit 419 includes a second airflow generation device (not illustrated), a flow path 412 having one end at which the second airflow generation device is provided, and a second opening 413 that is the other end of the flow path 412. These components form second airflow B4. The second airflow B4 controls the first airflows A4-1 and A4-2 containing an odor. The first openings 404-1 and 404-2 are inside the second opening 413. That is, the first openings 404-1 and 404-2 and the second opening 413 are provided so that movements of the first airflows A4-1 and A4-2 exiting the first openings 404-1 and 404-2 can be controlled by the second airflow B4.

The odor-containing airflow generation units 409-1 and 409-2 may be the same as the odor-containing airflow generation unit 109 described in (2) above, and description thereof also applies to the present example.

The control airflow generation unit 419 may be the same as the control airflow generation unit 419 described in (2) above, and description thereof also applies to the present example.

Types of odor components included in the odor carrying portion 403-1 and the odor carrying portion 403-2 may be the same or may differ.

When the types of odor components contained therein are the same, it is possible to adjust the intensity of the odor to be presented or adjust a range in which the odor is presented, for example, by generating the odor-containing airflow using only one of the odor-containing airflow generation units 409-1 and 409-2, or generating the odor-containing airflow using both of the odor-containing airflow generation units 409-1 and 409-2.

When the types of odor components contained therein differ, it is possible to switch the odor to be presented, for example, by switching the odor-containing airflow generation unit that generates the odor-containing airflow. Further, the odor-containing airflow is generated from both of the odor-containing airflow generation units, so that an odor in which two types of odor components are mixed is presented to the user.

Further, it is possible to promote mixing of airflows containing different odor components using the second airflow B4 generated by the control airflow generation unit in a case in which the types of odor components contained therein differ. Thus, the odor presentation module of the present technology can promote mixing of airflows containing different odor components in a plurality of odor containing airflow generation units.

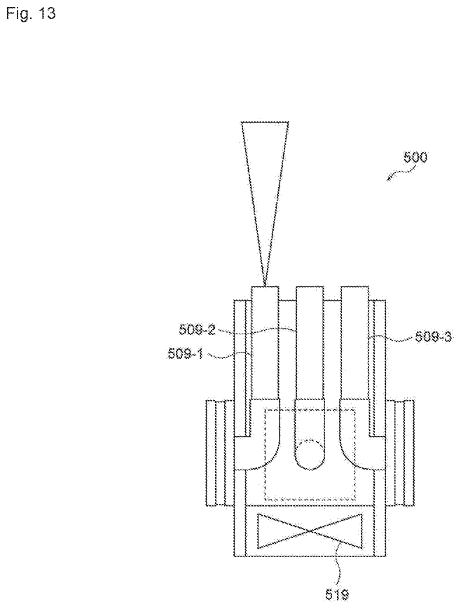

Further, the number of odor-containing airflow generation units included in the odor presentation module is not limited to two, and may be equal to or larger than three. The odor presentation module 500 according to the present technology illustrated in FIG. 13 includes three odor-containing airflow generation units 509-1, 509-2, and 509-3, and includes one control airflow generation unit. As illustrated in FIG. 13, all of the plurality of odor-containing airflow generation units may be provided in the flow path of the control airflow generation unit, or some of the plurality of odor-containing airflow generation units may be provided in the flow path of the control airflow generation unit and the remaining odor-containing airflow generation units may be provided outside the flow path of the control airflow generation unit.

(10) Ninth Example of First Embodiment (Example of Odor-Containing Airflow Generation Unit)

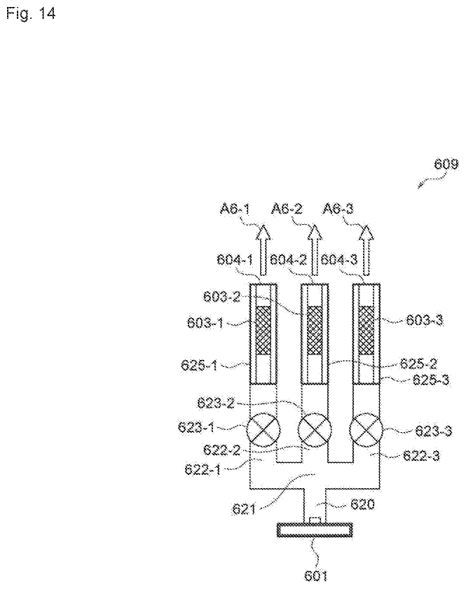

The odor containing airflow generation unit included in the odor presentation module according to the present technology may include one first airflow generation device and one odor carrying portion, may include one first airflow generation device and a plurality of odor carrying portions, may include a plurality of first airflow generation devices and one odor carrying portion, or may include a plurality of first airflow generation devices and a plurality of odor carrying portions. The odor-containing airflow generation unit described in (2) to (9) above includes one first airflow generation device and one odor carrying portion. Hereinafter, a configuration example of the odor-containing airflow generation unit including one first airflow generation device and a plurality of odor carrying portions will be described with reference to FIG. 14.

An odor-containing airflow generation unit 609 illustrated in FIG. 14 includes one first airflow generation device 601 and three odor carrying portions 603-1, 603-2, and 603-3. A flow path 620 connected to the first airflow generation device 601 is branched into three flow paths 622-1, 622-2, and 622-3 at a branch portion 621. Valves 623-1, 623-2, and 623-3 are provided in the flow paths 622-1, 622-2, and 622-3, respectively. Thus, the valves are provided in the respective branched flow paths, making it possible to select the odor carrying portion through which the airflow generated by the first airflow generation device 601 passes.

The flow paths 622-1, 622-2, and 622-3 include odor carrying portions 603-1, 603-2, and 603-3, respectively, and further, the flow paths 622-1, 622-2, and 622-3 have openings 604-1, 604-2, and 604-3 at respective ends. The odor-containing airflows A6-1, A6-2, and A6-3 exit the openings 604-1, 604-2, and 604-3. The odor carrying portions 603-1, 603-2, and 603-3 and the openings 604-1, 604-2, and 604-3 may be the same as the odor carrying portion 103 and the first opening 104 described in (2) above, and description thereof also applies to the present example.

Further, in the odor-containing airflow generation unit 609 illustrated in FIG. 14, a portion including the odor carrying portion and the opening (an area indicated by a thick line) can be replaced. More specifically, a portion 625-1 including the odor carrying portion 603-1 and the opening 604-1, a portion 625-2 including the odor carrying portion 603-2 and the opening 604-2, and a portion 625-3 including the odor carrying portion 603-3 and the opening 604-3 are configured to be replaceable. For example, these portions may be configured as a cartridge. The odor carrying portion is configured to be replaceable in this way, making it possible for a portion other than the odor carrying portion to be reused.

Types of odor components contained in the three odor carrying portions 603-1, 603-2, and 603-3 may be the same or may differ.

When the types of odor components contained in these are the same, it is possible to adjust the intensity of the odor to be presented or adjust a range in which the odor is presented, for example, by increasing or decreasing the number of valves to be opened.

When the types of odor components contained therein differ, it is possible to switch the odor to be presented, for example, by selecting a valve to be opened. Further, it is possible to present an odor in which two or three types of odor components are mixed to the user, by selecting the valve to be opened.

As described above, in the odor presentation module according to the present technology, the flow path through which the airflow generated by the first airflow generation device flows may be branched into two or more flow paths, and each of the branched flow paths may include an odor carrying portion.

2. Second Embodiment (Odor Presentation Device)

The present technology also provides an odor presentation device including the odor presentation module described in “1. First embodiment (odor presentation module)” above. The odor presentation device may include, for example, the odor presentation module and a housing for holding the odor presentation module.

Because the odor presentation device according to the present technology includes the odor presentation module, various expressions of the odor can be performed.

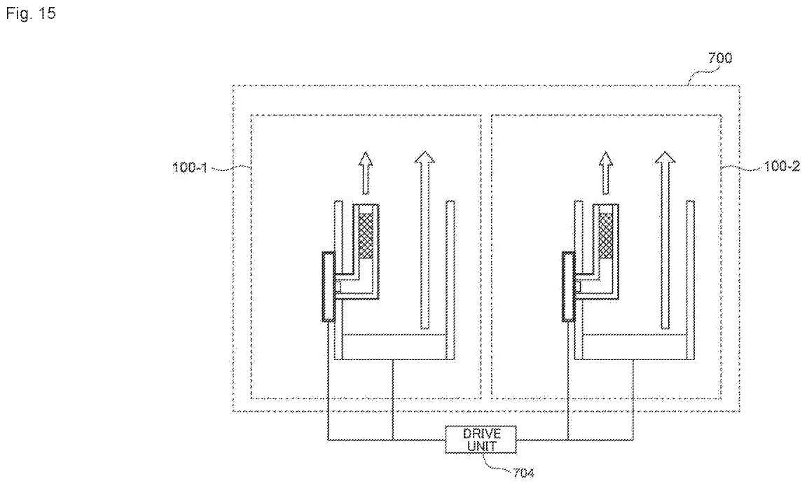

The odor presentation device according to the present technology may include one or more odor presentation modules. FIG. 15 illustrates a configuration example of an odor presentation device including two odor presentation modules. An odor presentation device 700 illustrated in FIG. 15 includes two odor presentation modules 100 described in (2) of “1. First embodiment (odor presentation module)” above.

The odor presentation device 700 may be connected to a drive unit 704 that drives the first airflow generation device 101 and the second airflow generation device 111 included in the two respective odor presentation modules 100 by a cable or wirelessly. Alternatively, the odor presentation device 700 may include the drive unit 704 therein. For example, control of the odor presentation module 100 by the drive unit 704 may be performed by a switch or drive software.

An example of the odor presentation device according to the present technology will be described hereinafter.

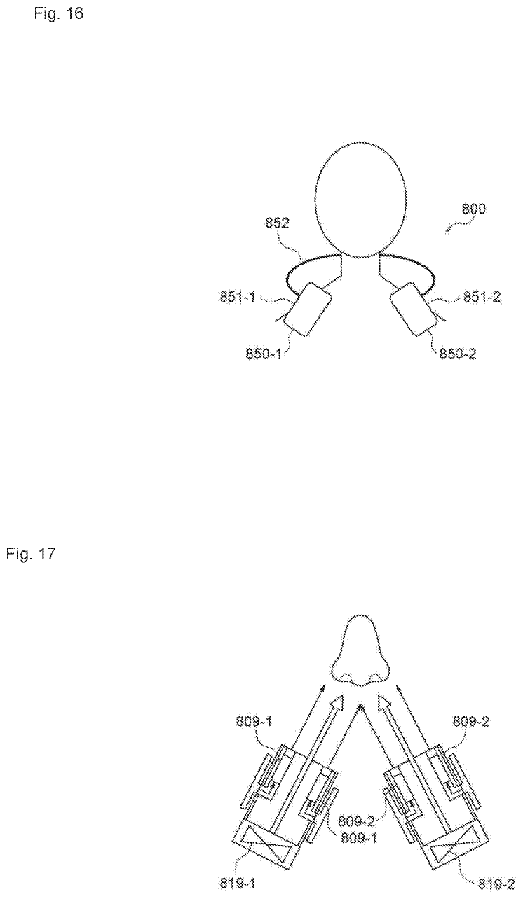

FIG. 16 illustrates a configuration example of the odor presentation device according to the present technology. An odor presentation device 800 illustrated in FIG. 16 includes odor presentation modules 850-1 and 850-2 according to the present technology, and a strap portion 852 having both ends to which these are connected. The odor presentation modules 850-1 and 850-2 may be held in housings 851-1 and 851-2, respectively, and may be stored in, for example, housings 851-1 and 851-2.

The odor presentation modules 850-1 and 850-2 are connected by the strap portion 852. With the strap portion 852, the user can use the odor presentation device 800, for example, by hanging the odor presentation device 800 around the neck. The strap portion 852 may include wirings that enable the odor presentation modules 850-1 and 850-2 to communicate with each other.

For example, each of the odor presentation modules 850-1 and 850-2 may be connected to a drive unit that drives these odor presentation modules by a cable or wirelessly. Alternatively, the odor presentation modules 850-1 and 850-2 may include the drive unit.

FIG. 17 illustrates a configuration example of the two odor presentation modules 850-1 and 850-2. As illustrated in FIG. 17, the odor presentation module 850-1 includes two odor-containing airflow generation units 809-1 and one control airflow generation unit 819-1. The odor presentation module 850-2 has the same configuration. This makes it possible to realize various ways of presenting an odor to a user, and further, to present a plurality of types of odors.

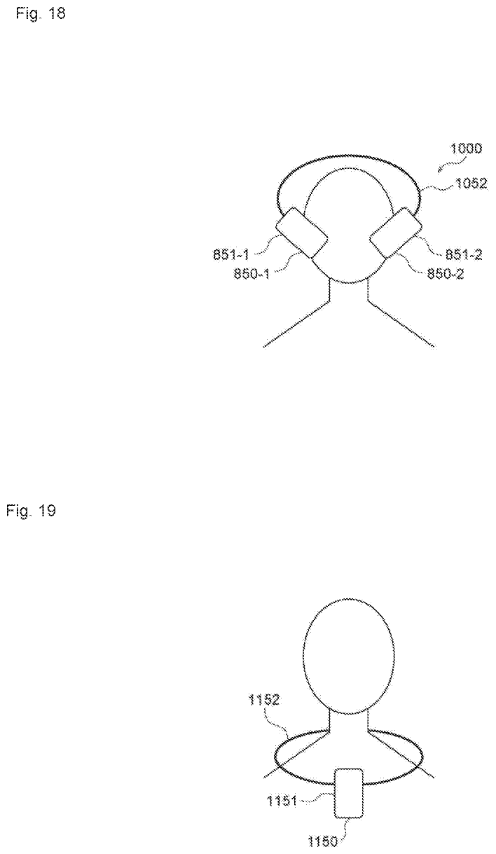

The odor presentation device including the two odor presentation modules is not limited to a device having a shape used by hanging the device around a neck. FIG. 18 illustrates another example of the odor presentation device according to the present technology. An odor presentation device 1000 illustrated in FIG. 18 includes two odor presentation modules 850-1 and 850-2, and a headband portion 1152 having both end to which the odor presentation modules 850-1 and 850-2 are connected. The headband portion 1152 allows the user to mount the odor presentation device 1000 on a head of the user, for example, and use the odor presentation device 1000. The headband portion 1152 may include wirings enabling the odor presentation modules 850-1 and 850-2 to communicate with each other.

FIG. 19 illustrates another configuration example of the odor presentation device according to the present technology. An odor presentation device 1100 illustrated in FIG. 19 includes an odor presentation module 1150 according to the present technology, and a strap portion 1052 to which the odor presentation module 1150 is connected. The strap portion 1052 has a necklace shape, and the strap portion 1052 is disposed in a pendant portion having a necklace shape. Thus, the odor presentation device according to the present technology may include one odor presentation module.

The odor presentation device according to the present technology may be configured to be connectable to a game controller, or may be configured to be connectable to an information processing device such as a smartphone or tablet.

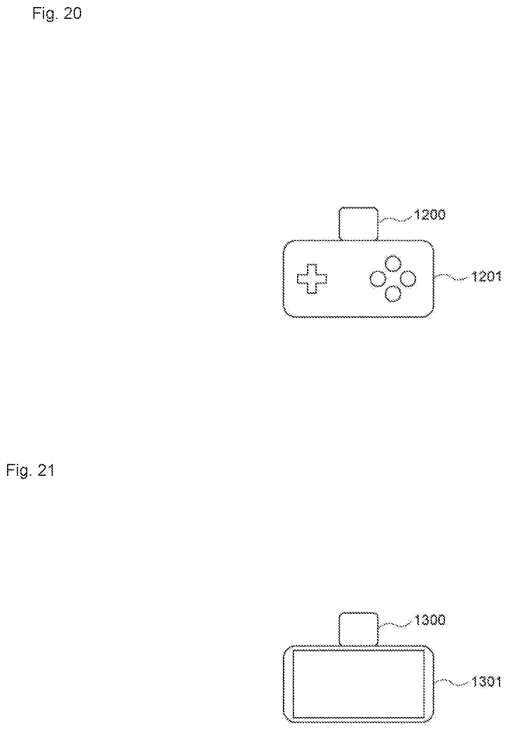

FIG. 20 illustrates an odor presentation device 1200 according to the present technology connected to a game controller 1201. The odor presentation device 1200 includes, for example, a connection terminal (not illustrated), and is connected to the game controller 1201 by the connection terminal. A type of connection terminal may be appropriately selected according to a type of game controller. The connection of the odor presentation device 1200 to the game controller 1201 makes it possible for an odor to be presented to a user playing a game. For example, an odor linked to a video that is viewed by the user playing the game may be presented to the user.

FIG. 21 illustrates an odor presentation device 1300 according to the present technology connected to a smartphone 1301. Also in this case, the odor presentation device 1300 includes, for example, a connection terminal (not illustrated), and is connected to the smartphone 1301 by the connection terminal. A type of connection terminal may be appropriately selected according to a type of smartphone. The connection of the odor presentation device 1300 to the smartphone 1301 makes it possible for an odor to be presented to a user watching a video or playing a game on the smartphone. For example, an odor linked to the video that is viewed by the user may be presented to the user.

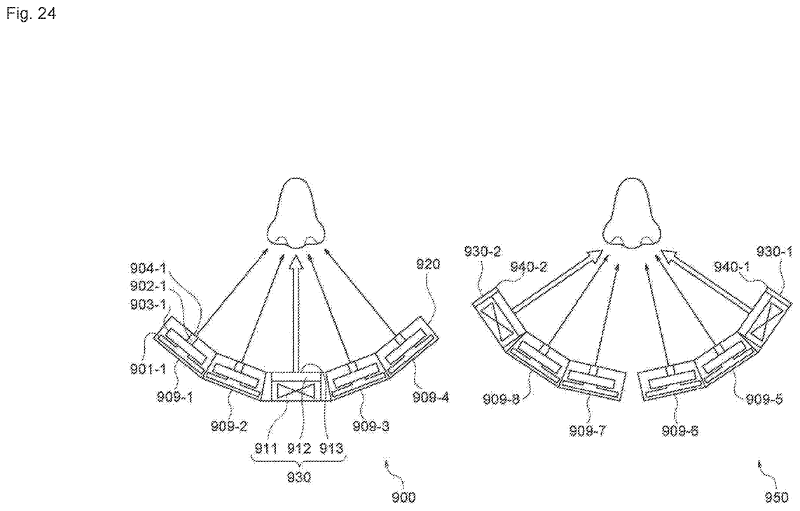

The odor presentation device according to the present technology may be configured to include a housing having one or a plurality of odor-containing airflow generation units and one or a plurality of control airflow generation units, for example. An example of this configuration will be described with reference to FIG. 24. Odor presentation devices 900 and 950 according to the present technology are shown on the left and right of FIG. 24, respectively.

The odor presentation device 900 shown on the left side of FIG. 24 includes four odor-containing airflow generation units 909-1, 909-2, 909-3, and 909-4, and one control airflow generation unit 930, and the four odor-containing airflow generation units and the one control airflow generation unit 930 are included in one housing 920.

The odor-containing airflow generation unit 909-1 includes a first airflow generation device 901-1, a flow path 902-1, an odor carrying portion 903-1, and a first opening 904-1. The other odor-containing airflow generation units 909-2, 909-3, and 909-4 have the same configuration. Odor components held by the odor carrying portions included in the plurality of respective odor-containing airflow generation units may be the same or may differ. Further, configurations of the plurality of odor-containing airflow generation units may be the same or may differ.

The control airflow generation unit 930 includes a second airflow generation device 911, a flow path 912, and a second opening 913.

The odor presentation device 950 shown on the right side of FIG. 24 includes a combination of a housing 940-1 including two odor-containing airflow generation units 909-5 and 909-6 and one control airflow generation unit 930-1, and a housing 940-2 including two odor-containing airflow generation units 909-7 and 909-8 and one control airflow generation unit 930-2. Thus, the odor presentation device according to the present technology may include two or more housings each including the odor-containing airflow generation unit and the control airflow generation unit.

The odor-containing airflow generation unit 909-5, 909-6, 909-7, and 909-8 may have the same configuration as the odor containing airflow generation unit 909-1 shown on the left side of FIG. 24. The odor components held by the odor carrying portions included in the plurality of respective odor-containing airflow generation units may be the same or may differ. Further, configurations of the plurality of odor-containing airflow generation units may be the same or may differ. The control airflow generation units 930-1 and 930-2 may have the same configuration as the control airflow generation unit 930 shown on the left side of FIG. 24. Configurations of these plurality of control airflow generation units may be the same or may differ.

3. Third Embodiment (Odor Presentation System)

The present technology also provides an odor presentation system including the odor presentation module described in “1. First embodiment (odor presentation module)” above or the odor presentation device described in “2. Second embodiment (odor presentation device)”. The odor presentation system may further include, for example, the odor presentation module or a control unit that controls the odor presentation device.

Because the odor presentation system according to the present technology includes the odor presentation module, various odor expressions can be realized.

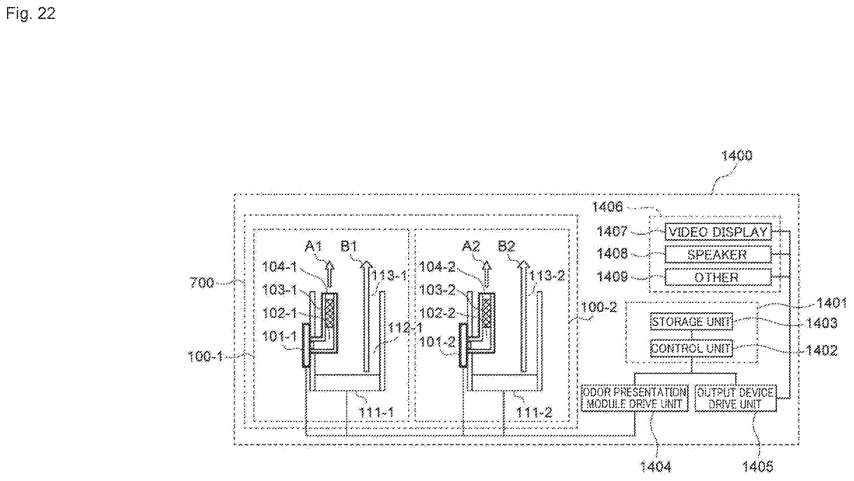

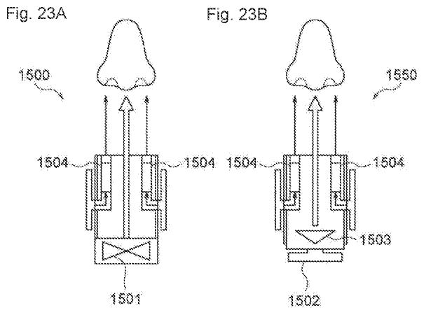

The odor presentation system according to the present technology may include one or a plurality of odor presentation modules. FIG. 22 illustrates a configuration example of an odor presentation system including two odor presentation modules. An odor presentation system 1400 illustrated in FIG. 22 includes two odor presentation modules 100-1 and 100-2, and these odor presentation modules may be the odor presentation module 100 described in (2) of “1. First embodiment (odor presentation module)” above, and description thereof also applies to the present embodiment.

The odor presentation system 1400 further includes an information processing device 1401, an odor presentation module drive unit 1404, an output device drive unit 1405, and an output device 1406. The information processing device 1401 includes, for example, a control unit 1402 and a storage unit 1403. The output device 1406 may include, for example, a video display 1407, a speaker 1408, and another output device 1409.

The information processing device 1401 includes, for example, a control unit 1402 and a storage unit 1403. The information processing device 1401 may be, for example, a computer, a smartphone, a tablet PC, a laptop personal computer, a desktop personal computer, a game machine, or a television, but is not limited thereto. The information processing device 1401 may be configured by, for example, a drive circuit.

The control unit 1402 controls the odor presentation module drive unit 1404 and the output device drive unit 1405. Under this control, the odor presentation module drive unit 1404 and the output device drive unit 1405 drive the odor presentation modules 100-1 and 100-2 and the output device 1409, respectively, so that presentation of the odor to the user and presentation of a video and audio are performed. Preferably, the control unit 1402 controls the odor presentation module drive unit 1404 and the output device 1406 so that the presentation of the odor is performed through link to the video or audio.

A function of the control unit 1402 may be realized by, for example, a central processing unit (CPU). Any processor can be used as the CPU 1002, and examples thereof can include Xeon (registered trademark) series, Core (trademark) series, or Atom (trademark) series of processors.

Video data, audio data, and data for the other output device, for example, may be stored in the storage unit 1403. Data regarding odor presentation associated with these pieces of data may be included, or data regarding odor presentation not associated with these pieces of data may be included in storage unit 1403. Further, a control program for driving the odor presentation module drive unit 1404 and/or the output device drive unit 1405 may be stored in the storage unit 1403. Further, a drive data creation program used for creation of data for driving the odor presentation modules 100-1 and 100-2 through link to video data and/or audio data may be stored in the storage unit 1403.

The storage unit 1403 may include, for example, a RAM and a storage medium. The RAM may include a cache memory and a main memory, and can temporarily store a program and the like that are used by the CPU. The storage medium may store an operating system (for example, WINDOWS (registered trademark), UNIX (registered trademark), or LINUX (registered trademark)), a program for realizing an odor presentation method according to the present technology to be described below, video data, audio data, data for the other output device, data regarding odor presentation, a control program for driving the odor presentation module drive unit and/or the output device drive unit, and a drive data creation program.

The odor presentation module drive unit 1404 drives the odor presentation modules 100-1 and 100-2 (particularly, the first and second airflow generation devices included therein) in response to a control signal from the control unit 1402. The control signal may be based on the data regarding the odor presentation.

The output device 1406 drives the output device 1409 in response to a control signal from the control unit 1402. The control signal may be based on, for example, the video data, the audio data, or data for the other output device.

The output device 1406 may include, for example, the video display 1407, the speaker 1408, and the other output device 1409. Examples of the other output device 1409 include a projector, a light, an illumination, a headphone, a vibration device, an actuator, an air pump, an air conditioner, and a bubble generator, but the other output device 1409 is not limited thereto.

The odor presentation system 1400 may further include an operating device and/or a sensing device. Examples of the operating device include a controller, a touch panel, a keyboard, and a mouse. Examples of the sensing device may include an acceleration sensor, a gyroscope, a geomagnetic sensor, an image sensor, and a vital sensor.

The information processing device 1401 may further include a communication device, a drive, and the like. The communication device connects the information processing device 1401 to a network by a cable or wirelessly. The communication device can acquire various types of data (for example, data regarding odor presentation, video data, audio data, and the like) via a network. The acquired data can be stored in, for example, a storage medium. The drive can read information recorded on the recording medium and output the information to the RAM. The recording medium is, for example, a microSD memory card, an SD memory card, or a flash memory, but is not limited thereto.

The odor presentation system 1400 may operate the odor presentation module (or the odor presentation device) in conjunction with, for example, the output device. This makes odor presentation linked to, for example, a video and/or an audio possible. That is, the odor presentation system 1400 can be used as a reproduction system that performs presentation of a video and/or an audio and odor presentation. That is, the present technology also provides a reproduction system that performs presentation of a video and/or an audio and performs presentation of an odor, which includes the odor presentation module according to the present technology.

The odor presentation system 1400 may be used as an editing system for creating a program or data for driving the odor presentation module (or the odor presentation device) and the output device in conjunction with each other. That is, the present technology also provides an editing system that includes the odor presentation module according to the present technology, and associates the data regarding on the odor presentation with video data and/or audio data. The odor presentation system 1400 used as the editing system can have, for example, an editing environment in which video data and/or audio data can be captured and a data file obtained by synchronizing an odor discharge timing with these pieces of data can be formed.

### 4. Fourth Embodiment (Odor Presentation Method)

The present technology also provides an odor presentation method. The odor presentation method includes a first airflow generation step in which first airflow containing an odor exits the first opening, and a second airflow generation step of generating second airflow exiting the second opening or second airflow entering the second opening, and movement of the first airflow exiting the first opening is controlled by the second airflow.

In the odor presentation method according to the present technology, the movement of the first airflow exiting the first opening is controlled by the second airflow as described above. Therefore, various methods of expressing an odor can be realized.

An order of the first airflow generation step and the second airflow generation step and a time at which these steps are performed may be appropriately set depending on the way the odor is presented. For example, the first airflow generation step may be performed first, and then the second airflow generation step may be performed, or these steps may be performed in the reverse order. Further, the first airflow generation step may be performed at a predetermined timing while the second airflow generation step is continuously performed, or the second airflow generation step may be performed at a predetermined timing while the first airflow generation step is continuously performed.

The method may be performed, for example, by the odor presentation system according to the present technology. Hereinafter, respective steps will be described with reference to FIG. 22.

In the first airflow generation step, the control unit 1402 controls the odor presentation module drive unit 1404 so that the odor presentation module 100-1 and/or 100-2 is driven. The odor presentation module drive unit 1404 drives the first airflow generation devices 101-1 and/or 101-2 of the odor presentation modules 100-1 and/or 100-2. The first airflow generation device 101-1 and/or 101-2 generates airflow, the airflow passes through the odor carrying portions 103-1 and/or 103-2, and the first airflow A1 and/or A2 containing an odor is generated. The first airflows A1 and/or A2 exits the first openings 104-1 and/or 104-2.

Thus, the odor presentation system 1400 exits the first airflows A1 and/or A2 containing an odor from the first openings 104-1 and/or 104-2.

In the second airflow generation step, the control unit 1402 controls the odor presentation module drive unit 1404 so that the odor presentation module 100-1 and/or 100-2 is driven. The odor presentation module drive unit 1404 drives the second airflow generation devices 111-1 and/or 111-2 of the odor presentation modules 100-1 and/or 100-2. The second airflow generation device 111-1 and/or 111-2 generates the second airflows B1 and/or B2. The second airflows B1 and/or B2 exits the second opening 113-1 and/or 113-2.

Thus, the odor presentation system 1400 emits the second airflows B1 and/or B2 containing an odor from the second openings 113-1 and/or 113-2.

The first openings 104-1 and 104-2 are inside the second openings 113-1 and 113-2, respectively. Therefore, movements of the first airflow A1 and A2 are controlled by the second airflow B1 and B2, respectively.

For example, the first airflow A1 is generated while the second airflow B1 is continuously generated, making it possible to express the drifting odor due to the odor component in the first airflow A1.

Further, it is possible to express the local odor by temporarily stopping the generation of the second airflow B1 and generating the first airflow A1 for a short time (for example, injecting the first airflow A1).

Further, it is possible to express the disappearance of the odor by continuously generating the first airflow A1 and stopping the generation of the first airflow A1 and generating the second airflow B1.

The method of expressing the odor is not limited to these. It is possible to realize various expressions of an odor by adjusting the flow velocity, a generation time, a generation start timing, a generation stop timing, and the like of the first airflow A1 and the second airflow B1.

Similarly, various expressions of the first airflow A2 and the second airflow B2 can be realized. Further, various expressions can be realized by various combinations of the first airflow A1 and the second airflow B1 and the first airflow A2 and the second airflow B2.

A more specific example of the odor expression in the odor presentation method in the present technology will be further described hereinafter.

Using the odor presentation method of the present technology, it is possible to present the odor to the user through link to, for example, a video indicating a situation in which an odor drifts softly or an experience in which an odor drifts softly. In this case, the odor presentation system 1400 of the present technology can be controlled as follows, for example.

First, the odor presentation system 1400 continuously generates the second airflows B1 and/or B2 (for example, a slightly weak airflow). Accordingly, the user does not receive wind pressure due to the second airflows B1 and/or B2.

The odor presentation system 1400 emits the first airflows A1 and/or A2 from the first openings 104-1 and/or 104-2 at a timing when the user views the video or receives the experience. Accordingly, the first airflows A1 and/or A2 is mixed with the second airflows B1 and/or B2 and reaches the user. Because a difference in wind pressure before and after the first airflows A1 and/or A2 reaches is small, it is possible to express a softly drifting odor.

Using the odor presentation method of the present technology, it is possible to present the odor to the user through link to a video or experience in which the odor is emitted to the user from a screen on which a video is presented (for example, a video or experience in which a spray can be applied, a video in which a cannon explodes to this side, or a video in which a pistol fires), for example, In this case, the odor presentation system 1400 of the present technology can be controlled as follows, for example.

First, the odor presentation system 1400 maintains its state when the odor presentation system 1400 stops the generation of the second airflows B1 and/or B2 or the generation of the second airflows B1 and/or B2 is not performed. Accordingly, the user does not receive the wind pressure due to the second airflows B1 and/or B2. At a timing when the user views the video or receives the experience, the odor presentation system 1400 emits the first airflows A1 and/or A2 from the first openings 104-1 and/or 104-2 (for example, the first airflows A1 and/or A2 are injected from the first openings 104-1 and/or 104-2). Accordingly, only the first airflows A1 and/or A2 reaches the user. Because a wind pressure difference before and after the first airflows A1 and/or A2 reaches is large, it is possible to express a suddenly generated odor.

Using the odor presentation method of the present technology, it is possible to express disappearance of the odor through link to a video or experience in which an odor on a screen on which a video is presented disappears (for example, a video or experience in which smoke disappears, or a video or experience in which a food generating an odor is sealed), for example. In this case, the odor presentation system 1400 of the present technology can be controlled as follows, for example.

The odor presentation system 1400 generates the first airflows A1 and/or A2. Accordingly, the user perceives the odor. The second airflows B1 and/or B2 may not be generated or may be generated while the first airflows A1 and/or A2 are generated. Then, the odor presentation system 1400 stops the generation of the first airflows A1 and/or A2. According to the stop timing, the odor presentation system 1400 starts the generation of the second airflows B1 and/or B2 when the second airflows B1 and/or B2 is not generated, or increases an air volume of the second airflows B1 and/or B2 when the second airflows B1 and/or B2 is generated. This makes it possible for the user not to perceive the odor component contained in the first airflows A1 and/or A2, that is, express the disappearance of the odor.

Further, the odor presentation system 1400 may reverse directions of the second airflows B1 and/or B2 from the outside of each odor presentation module to each second airflow generation device according to the stop timing. That is, the odor presentation system 1400 may start intake using the second airflow generation device. This makes it possible for the user not to perceive the odor component contained in the first airflows A1 and/or A2, that is, express the disappearance of the odor.

The intake enables recovery and/or discharge of the odor component remaining in the odor presentation module or the odor component leaking from the first opening, and contributes to preventing an unnecessary odor from reaching the user.

The way the odor is expressed in the odor presentation method of the present technology is not limited to the above examples.

For example, the odor presentation system 1400 causes the second airflows B1 and/or B2 to be generated, and stops the generation of the second airflows B1 and/or B2 at the same time as start of the generation of the first airflows A1 and/or A2 (particularly, generation during a short time and, more specifically, injection). Then, the odor presentation system 1400 stops the generation of the first airflows A1 and/or A2 and restarts the generation of the second airflows B1 and/or B2. This makes it possible to express, for example, a temporary odor generation (for example, a temporary odor generation).

Further, the odor presentation system 1400 can adjust the flow velocities of the first airflows A1 and A2 and the second airflows B1 and B2. For this adjustment, the control unit 1402 of the odor presentation system 1400 controls the odor presentation module drive unit 1404 to adjust the flow velocity of the airflow generated by the first airflow generation devices 101-1 and 101-2 and the second airflow generation devices 111-1 and 111-2. This makes it possible to change, for example, the intensity of the odor caused by the first airflow A1 and A2, a mixing ratio of the first airflow A1 and A2, a mixing ratio of the first airflow A1 and the second airflow B1, a mixing ratio of the first airflow A2 and the second airflow B2, wind pressure felt by the user, and the like.

The present technology can also have the following configurations.

[1] An odor presentation module including:

a first opening that first airflow containing an odor exits; and

a second opening that second airflow exits,

wherein the first opening and the second opening are provided so that movement of the first airflow exiting the first opening is able to be controlled by the second airflow.

[2] The odor presentation module according to [1],

wherein the odor presentation module includes a first airflow generation device, and an odor carrying portion that carries the odor, and

the odor presentation module is configured such that airflow generated by the first airflow generation device and the odor included in the odor carrying portion form the first airflow.

[3] The odor presentation module according to [1] or [2], further including a second airflow generation device, wherein the second airflow generation device generates the second airflow.

[4] The odor presentation module according to any one of [1] to [3], configured such that the first airflow is injected from the first opening.

[5] The odor presentation module according to any one of [1] to [4], configured such that the first airflow and the second airflow come into contact with each other outside the odor presentation module.

[6] The odor presentation module according to any one of [1] to [5], configured such that the first airflow and the second airflow come into contact with each other inside the odor presentation module.

[7] The odor presentation module according to any one of [1] to [6], wherein the first opening is in front of the second airflow generation device in a traveling direction of the second airflow.

[8] The odor presentation module according to any one of [1] to [7], configured to be able to change the flow velocity of at least one of the first airflow and the second airflow.

[9] The odor presentation module according to any one of [1] to [8], configured to be able to change a direction of the second airflow to a direction into the second opening.

[10] An odor presentation module including:

an odor-containing airflow generation unit; and

a control airflow generation unit configured to control odor containing airflow generated by the odor-containing airflow generation unit,

wherein the odor-containing airflow generation unit includes a first airflow generation device, an odor carrying portion through which airflow generated by the first airflow generation device passes, and a first opening that first airflow containing an odor formed by passing through the odor carrying portion exits, and

the control airflow generation unit includes a second airflow generation device, and a second opening that second airflow generated by the second airflow generation device exits.

[11] The odor presentation module according to [10], wherein the first opening and the second opening are provided so that movement of the first airflow exiting the first opening is able to be controlled by the second airflow.

[12] The odor presentation module according to [10] or [11], including a first flow path through which the first airflow flows, and a second flow path through which the second airflow flows,

wherein the first flow path is provided inside the second flow path, or is provided outside the second flow path.

[13] The odor presentation module according to any one of [10] to [12], wherein the odor carrying portion is configured to be replaceable.

[14] The odor presentation module according to any one of [10] to [13], wherein a flow path through which the airflow generated by the first airflow generation device flows is branched into two or more flow paths, and each of the branched flow paths has the odor carrying portion.

[15] The odor presentation module according to any one of [10] to [14], wherein an opening and closing mechanism for controlling the passage of airflow is provided in one or both of an inlet through which the airflow generated by the first airflow generation device enters the odor carrying portion and an outlet or the first opening through which first airflow containing an odor exits the odor carrying portion.

[16] The odor presentation module according to any one of [10] to [15], configured to curb the generation of airflow in the flow path through which the first airflow flows at the time of stopping of the first airflow generation device.

[17] An odor presentation device including:

an odor presentation module including a first opening that first airflow containing an odor exits, and a second opening that second airflow exits, the first and second openings being provided so that movement of the first airflow exiting the first opening is able to be controlled by the second airflow; and

a housing configured to hold the odor presentation module.

[18] An odor presentation system including:

an odor presentation module including a first opening that first airflow containing an odor exits, and a second opening that second airflow exits, the first and second openings being provided so that movement of the first airflow exiting the first opening is able to be controlled by the second airflow; and

a control unit configured to control the odor presentation module.

[19] An odor presentation method including:

a first airflow generation step of emitting first airflow containing an odor from a first opening; and

a second airflow generation step of generating second airflow exiting a second opening or second airflow entering the second opening,

wherein movement of the first airflow exiting the first opening is controlled by the second airflow.

### REFERENCE SIGNS LIST

100, 100-1 to 100-6 Odor presentation module

101, 101-1, 101-2 First airflow generation device

102, 152, 162 Flow path

103, 163 Odor carrying portion

104, 154, 164 First opening

105, 155 Inlet

106, 156 Outlet

109, 109-1 to 109-6 Odor-containing airflow generation unit

111 Second airflow generation device

112 Flow path

113 Second opening

119 Control airflow generation unit

121, 131 Discharge port

122, 132 Intake port

123, 124, 133, 143 Opening and closing valve

125 Diaphragm

126 Piezoelectric element

141, 142 Solenoid valve

144 Shield

157 Check valve

1. An odor presentation module comprising:
a first opening that first airflow containing an odor exits; and
a second opening that second airflow exits,
wherein the first opening and the second opening are provided so that movement of the first airflow exiting the first opening is able to be controlled by the second airflow.

2. The odor presentation module according to claim 1,
wherein the odor presentation module includes a first airflow generation device, and an odor carrying portion that carries the odor, and
the odor presentation module is configured such that airflow generated by the first airflow generation device and the odor included in the odor carrying portion form the first airflow.

3. The odor presentation module according to claim 1, further comprising a second airflow generation device, wherein the second airflow generation device generates the second airflow.

4. The odor presentation module according to claim 1, configured such that the first airflow is injected from the first opening.

5. The odor presentation module according to claim 1, configured such that the first airflow and the second airflow come into contact with each other outside the odor presentation module.

6. The odor presentation module according to claim 1, configured such that the first airflow and the second airflow come into contact with each other inside the odor presentation module.

7. The odor presentation module according to claim 3, wherein the first opening is in front of the second airflow generation device in a traveling direction of the second airflow.

8. The odor presentation module according to claim 1, configured to be able to change the flow velocity of at least one of the first airflow and the second airflow.

9. The odor presentation module according to claim 1, configured to be able to change a direction of the second airflow to a direction into the second opening.

10. An odor presentation module comprising:
an odor-containing airflow generation unit; and
a control airflow generation unit configured to control odor-containing airflow generated by the odor-containing airflow generation unit,
wherein the odor-containing airflow generation unit includes a first airflow generation device, an odor carrying portion through which airflow generated by the first airflow generation device passes, and a first opening that first airflow containing an odor formed by passing through the odor carrying portion exits, and
the control airflow generation unit includes a second airflow generation device, and a second opening that second airflow generated by the second airflow generation device exits.

11. The odor presentation module according to claim 10, wherein the first opening and the second opening are provided so that movement of the first airflow exiting the first opening is able to be controlled by the second airflow.

12. The odor presentation module according to claim 10, comprising a first flow path through which the first airflow flows, and a second flow path through which the second airflow flows,
wherein the first flow path is provided inside the second flow path, or is provided outside the second flow path.

13. The odor presentation module according to claim 10, wherein the odor carrying portion is configured to be replaceable.

14. The odor presentation module according to claim 10, wherein a flow path through which the airflow generated by the first airflow generation device flows is branched into two or more flow paths, and each of the branched flow paths has the odor carrying portion.

15. The odor presentation module according to claim 10, wherein an opening and closing mechanism for controlling the passage of airflow is provided in one or both of an inlet through which the airflow generated by the first airflow generation device enters the odor carrying portion and an outlet or the first opening through which first airflow containing an odor exits the odor carrying portion.

16. The odor presentation module according to claim 10, configured to curb the generation of airflow in the flow path through which the first airflow flows at the time of stopping of the first airflow generation device.

17. An odor presentation device comprising:
an odor presentation module including a first opening that first airflow containing an odor exits, and a second opening that second airflow exits, the first and second openings being provided so that movement of the first airflow exiting the first opening is able to be controlled by the second airflow; and
a housing configured to hold the odor presentation module.

18. An odor presentation system comprising:
an odor presentation module including a first opening that first airflow containing an odor exits, and a second opening that second airflow exits, the first and second openings being provided so that movement of the first airflow exiting the first opening is able to be controlled by the second airflow; and
a control unit configured to control the odor presentation module.

19. An odor presentation method comprising:
a first airflow generation step of emitting first airflow containing an odor from a first opening; and
a second airflow generation step of generating second airflow exiting a second opening or second airflow entering the second opening,
wherein movement of the first airflow exiting the first opening is controlled by the second airflow.
